(19) United States
(12) Patent Application Publication
(10) Pub. No.: US 2018/0238876 A1
(43) Pub. Date: Aug. 23, 2018

(54) Method For Assessing Cell Surface Receptors of Blood Cells

(71) Applicant: GENTIAN DIAGNOSTICS AS, Moss (NO)

(72) Inventor: Erling Sundrehagen, Mellerud (SE)

(21) Appl. No.: 15/741,456

(22) PCT Filed: Jul. 25, 2016
(86) PCT No.: PCT/EP2016/067639

§ 371 Date: Jan. 2, 2018

(30) Foreign Application Priority Data
Jul. 23, 2015 (NO) 20150960

(51) Int. Cl. G01N 33/569; G01N 33/543

(52) U.S. Cl. CPC G01N 33/56972; G01N 33/54386

(57) ABSTRACT

The present invention relates to a novel method for rapid assessment of one or more subclasses of blood cells of interest (BCol), as for example CD4+ cells and CD8+ cells, in a liquid whole blood sample or a sample derived therefrom; a method of determining the cell count for such cells; a method for determining the CD4/CD8 ratio; a method for determining the quantity of such receptors in a sample; as well as a vertical flow assay device for performing such assessment.

The present invention relates to a novel method for rapid assessment of one or more subclasses of blood cells of interest (BCol), as for example CD4+ cells and CD8+ cells, in a liquid whole blood sample or a sample derived therefrom; a method of determining the cell count for such cells; a method for determining the CD4/CD8 ratio; a method for determining the quantity of such receptors in a sample; as well as a vertical flow assay device for performing such assessment.

### BACKGROUND OF THE INVENTION

Whole blood is a term used for human blood from a standard blood donation or blood sampling. The blood is typically combined with an anticoagulant during the collection process, but is generally otherwise unprocessed. Whole blood comprises the blood plasma, red blood cells (erythrocytes) and white blood cells (leucocytes) and platelets.

Heparin, citrate and EDTA (Ethylene Diamine Tetra Acetic Acid) are commonly used anticoagulation agents added to hinder coagulation in blood samples for laboratory analytical use.

CD4+ T helper cells are white blood cells that are an essential part of the human immune system. They are often referred to as CD4 cells, T-helper cells or T4 cells, and are a subpopulation of lymphocytes. They are called helper cells because one of their main roles is to send signals to other types of immune cells, including CD8 killer cells. CD4 cells send the signal and CD8 cells destroy the infectious particle. If CD4 cells become depleted, for example in untreated HIV infection, or following immune suppression prior to a transplant, the body is left vulnerable to a wide range of infections that it otherwise would have been able to fight.

The blood cells comprise often cell surface receptors (membrane receptors, often in the form of transmembrane receptors). These molecules are specialized integral membrane proteins that take part in communication between the cell and the outside world. Extracellular signaling molecules (usually hormones, neurotransmitters, cytokines, growth factors or cell recognition molecules) attach to the receptor, triggering changes in the function of the cell. This process is called signal transduction: The binding initiates a chemical change on the intracellular side of the membrane. In this way the receptors play a unique and important role in cellular communications and signal transduction. Many transmembrane receptors are composed of two or more protein subunits which operate collectively and may dissociate when ligands bind, fall off, or at another stage of their “activation” cycles. (Wikipedia citation Jul. 24, 2014).

The receptors called CD4 (cluster of differentiation 4) are glycoproteins found on the surface of immune cells such as T helper cells, monocytes, macrophages, and dendritic cells. CD4 receptors were discovered in the late 1970s and were originally known as leu-3 and T4 (after the OKT4 monoclonal antibody that reacted with it) before being named CD4 in 1984. In humans, the CD4 protein is encoded by the CD4 gene. (Isobe M, Huebner K, Maddon P J, Littman D R, Axel R, Croce C M (June 1986). “The gene encoding the T-cell surface protein T4 is located on human chromosome 12”. Proc. Natl. Acad. Sci. U.S.A. 83 (12): 4399-4402, and Ansari-Lari M A, Muzny D M, Lu J, Lu F, Lilley C E, Spanos S, Malley T, Gibbs R A (April 1996). “A gene-rich cluster between the CD4 and triosephosphate isomerase genes at human chromosome 12p13”. Genome Res. 6 (4): 314-26.)

HIV infection leads to a progressive reduction in the number of T cells expressing CD4. Medical professionals refer to the “CD4 count” to decide when to begin treatment during HIV infection or how to regulate medication during disease. Normal blood values are usually expressed as the number of cells per microliter (μL) (or cubic millimeter, mm³) of blood, with normal values for CD4 cells being 500-1200 cells/mm³. A CD4 count measures the number of T cells expressing CD4. While CD4 counts are not a direct HIV test—e.g. they do not check the presence of viral DNA, or specific antibodies against HIV—they are used to assess the immune system of a patient. Patients often undergo treatments when the CD4 counts reach a level of 350 cells/μL in Europe but usually around 500 cells/μL in the US; people with less than 200 cells/μL are at high risk of contracting AIDS defined illnesses. The newest National Institute of Health guidelines recommend treatment of any HIV-positive individuals, regardless of CD4 count. Medical professionals also refer to CD4 tests to determine efficacy of treatment.

Not only T helper cells carry surface and cytoplasmic CD4 receptors. In J Immunol Methods. 1990 Dec. 31; 135(1-2):59-69, Filion et al. reported that all monocytes are CD4 positive. The number of monocytes in whole blood is generally high. A method to determine the number of CD4 receptors associated with T helper cells therefore needs to encompass a step or a part of the method sorting away monocytes also carrying CD4 receptors.

Other blood cells carrying CD4 (like macrophages) are contained in low, and in this context neglectable proportions in the blood.

Flow cytometry is a powerful tool for identifying and enumerating cells. The flow cytometer detects and counts individual cells passing in a stream through a laser beam. By examining large numbers of cells, flow cytometry can give quantitative data on the percentage of cells bearing different molecules, such as surface immunoglobulin, which characterizes B cells, the T-cell receptor-associated molecules known as CD3, and the CD4 and CD8 co-receptor proteins that distinguish the major T-cell subsets. Individual cells within a mixed population are tagged with specific antibodies labelled with fluorescent dyes, or for example, by specific antibodies followed by labelled anti-immunoglobulin antibodies. The suspended mixture of labelled cells is then forced through an aperture, creating a fine stream of liquid containing cells spaced singly at intervals. As each cell passes through a laser beam it scatters the laser light, and any dye molecules bound to the cell will be excited and will fluoresce. Sensitive photomultiplier tubes detect both the scattered light, which gives information on the size and granularity of the cell, and the fluorescence emissions, which give information on the binding of the labelled antibodies and hence on the expression of cell-surface proteins by each cell. If two or more antibodies are used, each coupled to a different fluorescent dye, then the data may be displayed in the form of a two-dimensional scatter diagram or as a contour diagram, where the fluorescence of one dye-labelled antibody is plotted against that of a second, with the result that a population of cells labelling with one antibody can be further subdivided on the basis of its reactivity with the second antibody.

Typically, CD4 counts are measured in laboratories using said flow cytometry technology. Expensive and sophisticated equipment is needed, as well as highly trained personnel, a clean water supply and cold chain storage for reagents is generally required, necessitating the test to be carried out in centralized locations. Delays between testing and obtaining results can also lead to a significant ‘loss to follow up’ of patients and often they do not return to receive life-saving treatment.

In addition, the majority of non-reference laboratories and clinics in countries most affected by HIV cannot regularly monitor CD4 counts and access to testing can be difficult or even impossible in rural areas.

To facilitate near-patient testing and reduce the need for centralized laboratories with very advanced and complicated flow cytometer instruments, the Alere Inc, US, has developed the so-called PIMA system; see “Evaluation of the PIMA Point-of-Care CD4 Analyzer in VCT Clinics in Zimbabwe” by Sekesai Mtapuri-Zinyowera et al. in J Acquir Immune Defic Syndr 2010; 55:1-7. Therein the following is stated: For PIMA testing, each participant provided 1-2 drops of blood by lancet finger stick that were collected directly from the fingertip into the PIMA CD4 cartridge. A puncture depth of 1.8 mm with a blade-type lancet (Sarstedt) was used to achieve sufficient capillary blood flow. The PIMA cartridge collected the blood in a 25 μL receptacle. Of this initial volume, 5 μL of blood was drawn into the PIMA cartridge and further used for cytometric analysis. The cartridge was capped and inserted immediately into the PIMA analyzer to run the test. During the analysis process, the blood was automatically mixed with freeze-dried fluorescently labeled antibodies (anti-CD3 and anti-CD4) contained in the cartridge and transferred to a detection chamber where images were taken of the labeled cells to calculate the number of CD4 cells per mL of blood.”

The PIMA system was a good progress for near-patient testing, but still the PIMA system is based on a sophisticated instrument comprising a complex cassette which is expensive in production.

Immunoassays are another particularly useful form of assay that exploit the specificity, strength and diversity of antibody-antigen reactions to analyze samples and detect specific components therein. A wide range of immunoassay techniques is available, such as those described in “The Immunoassay Handbook” Nature Publishing Group, 2001. A wide range of methods for the detection of antibodies to specific antigens is also known. For example, the enzyme-linked immunosorbent assay (ELISA) or the radio-immunoassay (RIA) is routinely used in laboratories. Arrays and high-throughput screening methods are also employed. These methods generally require a high level of skill in laboratory techniques. A variety of methods has also been developed which require little skill and are rapid to perform, and which are therefore suitable for the detection of antibody to specific antigens, and/or the detection of specific antigens, at the point of care. In particular, lateral flow, dipstick and capillary tube kits have been developed to assay for a number of infections including viral infections.

In one method of detecting CD4 cells, dynabeads coated with anti-CD4 antibodies are used to bind CD4+ T-lymphocytes. Monocytes, that express CD14 and CD4, are excluded from fresh blood samples sample using beads coated with anti-CD14 antibodies. Reference is made to the publication “T regulatory-1 cells induce IgG4 production by B cells: role of IL-10” by Satoguina J S, Weyand E, Larbi J, Hoerauf A, in J Immunol (2005) 174:4718-4726. Thereafter, the isolated CD4 T-lymphocytes are lysed, stained with acridine orange and stained nuclei are enumerated by fluorescence microscopy.

A “TRAx CD4” test kit is described in Paxton et al., Clin. Diagn. Lab. Immunol., 2(1):104-114, 1995. This kit is an ELISA based method to measure total CD4 in whole blood samples. The antibodies used did not distinguish between cell-bound and soluble CD4 (see Lyamuya et al., J. Imm Methods, 195:103-112, 1996).

WO 2006/115866 describes an immunochromatographic device for measuring CD4 antigens. However, again there is no disclosure in this document of a capture reagent capable of distinguishing between cell-bound and soluble CD4 lacking a cytoplasmic domain in sample from a subject. Further, the device described in WO 2006/115866 depends upon the flow of sample over a series of numbered capture areas to capture CD4 by saturating consecutive capture areas on a test strip to subsequently provide a visual indication of the concentration of CD4 cells in the sample.

In an illustrative embodiment, the method is used for evaluating in a blood sample from a subject the level of T-cell associated CD4 comprising a cytoplasmic (cytosolic) and an extracellular (ecto) domain or the level of CD4 T-cells, the method comprising:
(i) optionally contacting the sample with an agent capable of lysing or permeabilizing CD4 T-cells;
(ii) contacting the sample with an antibody or antigen-binding fragment thereof that binds to the cytoplasmic domain of CD4; and
(iii) directly or indirectly evaluating the level or presence of bound CD4 in the sample.

Anderson et al in U.S. Pat. No. 8,409,818 describe a lateral flow method for evaluating from a subject the level of T-cell associated CD4 comprising a cytoplasmic (cytosolic) and an extracellular (ecto) domain or the level of CD4 T-cells. Said method comprises:
a) applying the test sample to a sample portion of an immunochromatographic device wherein the sample portion is operably connected to a capture portion of the device and wherein components of the test sample flow from the sample portion to and through the capture portion which comprises an antibody or antigen-binding fragment thereof that binds to the cytoplasmic domain of CD4 such that only CD4 comprising a cytoplasmic domain and not soluble CD4 that does not comprise a cytoplasmic domain binds to the antibody or fragment thereof to form a captured CD4;
b) contacting the capture portion with a second binding agent that binds to CD4 including the cytoplasmic or extracellular domain and which comprises a detection marker or which is capable of binding to a third or subsequent binding partner comprising a detection marker; and
c) optionally contacting the second binding agent with a third or subsequent binding agent comprising a detection marker; evaluating the presence of the detection marker.

This technology has been developed into the commercial product “VISITECT® CD4”, produced by Omega Diagnostics, UK. The device which is a disposable, near-patient test device for the determination of CD4, comprising both the part of the CD4 receptor exposed on the surface and the intracellular part of the CD4 receptor of the cells. In this way, co-measurement of soluble parts of the CD4 receptor present in the blood plasma and not bound to the white blood cells is avoided. Furthermore, a magnetic separation of monocytes is an integral part of the test device. The test is easy-to-use and only requires a finger-prick blood sample to perform the test. It is a test device that is well suited for testing where sophisticated laboratory equipment is not available. However, it is a time consuming 40-minutes-test, which also requires a manual addition of a separate liquid reagent after 17 minutes, which opens for operator induced faults in the processing of the test. Furthermore, a complicated production with a built-in magnetic separation device and magnetic particles for processing the separation step is needed.

In lateral flow technology, the flow of reagents and sample is parallel to the surface of the device, typically a filtration device, with reagents—often in dry form—is connected to or placed or immobilized within the filter. Numerous such test devices have been made, both for qualitative, semi-quantitative and quantitative measurement of high number of analytes. In Anal Bioanal Chem (2009) 393:569-582, Geertruida A. Posthuma-Trumpie & Jakob Korf & Aart van Amerongen provide a review entitled “Lateral flow immunoassay: its strengths, weaknesses, opportunities and threats.”

In general, an alternative technology to lateral flow technology can be a vertical flow technology. Corresponding products have been made where sample and reagents are vertically passed through filtration devices, optionally with specific binders immobilized in the filter. Tests for antibodies against HIV viruses have been made e.g. by Medmira Inc, Canada, using immobilized antigen or antigen fragments in the filter device, as described by Owen et al in Journal of Clinical Microbiology, May 2008, p. 1588-1595 Vol. 46, No. 5 entitled “Alternative Algorithms for Human Immunodeficiency Virus Infection Diagnosis Using Tests That Are Licensed in the United States.”

The NycoCard CRP test is a 2-minute Point of Care test to indicate bacterial or viral cause of infection. NycoCard CRP measures C-reactive protein (CRP), an acute phase protein that increases rapidly after onset of infection, as described in “Evaluation of a near-patient test for C-reactive protein used in daily routine in primary healthcare by use of difference plots” by Dahler-Eriksen et al. in Clinical Chemistry November 1997 vol. 43 no. 11 2064-2075. The vertical flow assay is characterized by a sample volume of 5 μL, an assay time of 2 minutes, sample material of whole blood, serum or plasma, measuring range: 8-200 mg/L for whole blood samples and 5-120 mg/L for serum and plasma samples. There is no sample crossover, it employs a disposable test device with immobilized antibodies to CRP in a nitrocellulose filtration membrane, and uses gold colloid particles conjugated to anti-CRP antibodies. There is low risk of sample contact, and a simple reflectometric reading of the color on the membrane surface is correlated to CRP concentration in the sample which is tested.

There is a need for a simple and fast, low cost method and device for the assessment of cell surface receptors specific for a subclass of blood cells of diagnostic or therapeutic interest, like for example T-cell associated CD4 receptors, in whole blood samples.

### SUMMARY OF THE INVENTION

The above problem was surprisingly solved by the methods and devices as defined in the claims and described in more detail herein below.

The present invention very surprisingly made it possible to apply for the assessment of specific blood cells the fast vertical flow principle, and, specifically, without any use of immobilized specific binder in the filter. The present invention employs the use of colorimetric measurement of the color developed on the surface of a filter, which is well known in vertical flow immunoassays, however antibody immobilization in the filter is not necessary.

The present invention relates in one embodiment to a method for the assessment of the amount of receptor molecules of a specific class of receptors bound to a “specific class” or “specific group” of cells (also designated as blood cells of interest (BCol)) in a sample of whole blood or a sample derived from whole blood. Typically, said class of receptor molecules is the class of CD4 receptors, and typically, the class (also designated herein as subclass, subset or subpopulation) of cells carrying said receptor molecules are T-lymphocytes.

The assay method of the present invention is characterized by mixing in a “first step” the said sample or an aliquot of the said sample with a “first liquid” comprising antibodies binding to other structures on the surface of “other cells” different from said abovementioned specific group (or class) of cells but carrying said receptors (said “other cells” are also designated as disturbing blood cells (DBC)), forming particles or aggregates or clusters of particles or cells with a size significantly larger than the size of the cells in said specific group of cells.

In one embodiment of the present invention, the said antibodies in said “first liquid” have a specific affinity for CD14, which is very abundant on monocytes (and monocytes also carry CD4 receptors, and thus would otherwise disturb the assay).

In some embodiments the method of the present invention is further characterized by said “first liquid” carrying antibodies towards said “other cells” forming clusters of said “other cells” or the antibodies are polymerized or immobilized on particles or polymers or other large molecules facilitating the formation of particles or aggregates or clusters of particles or cells with a size significantly larger than the size of the cells in “said specific group” of cells.

In one embodiment of the present invention, the antibodies of said “first liquid” have a specific affinity for receptor CD14, thus enabling the formation of particles or aggregates or clusters of particles or cells comprising the monocytes of the said sample with a size significantly larger than the size of the cells in said “specific group” of cells. Preferentially, the said “first liquid” has a low ionic strength, and a high enough volume to maintain the said low ionic strength after being mixed with the sample to be analyzed, to rapidly lyse the erythrocytes of the sample.

Hypotonic lysis of erythrocytes without lysis of leucocytes is described by Cunha et al in Anal. Methods, 2014, 6, 1377-1383, entitled “Kinetics of hypotonic lysis of human erythrocytes”. Lysis of the erythrocytes is very common in vertical flow immunoassays of whole blood samples.

The “second step” of the method of the present invention is a filtration step where the said particles or clusters or aggregates, with significantly larger size than the “specific group” of cells to be analyzed, are filtered away with a first filter letting the “specific group” of cells to be analyzed through the filter.

In one embodiment of the invention, cells comprising CD14 receptors, including the monocytes of the sample, having formed clusters by reacting with antibodies with specific reactivity towards CD14 receptors—optionally with antibodies being conjugated to polymers or immobilized on particles—are in this way filtered away using a filter letting T cells (and T cells carrying CD4 receptors) through the filter. The said “first filter” must have a pore size which enables the passage of the “specific group” of cells to be analyzed. In an embodiment of the invention, the said “specific group” of cells is constituted by T-lymphocytes, hence—in the said embodiment—the said “first filter” must have a pore-size enabling the T-lymphocytes to pass through the filter.

Filter materials having a low unspecific binding of cells and having a rather narrow distribution of pore sizes is preferred. Nylon web filters is a very good option, since it pore-size is well defined, and it's unspecific binding is rather low. T-lymphocytes will easily pass through a 30 μM filter, however aggregates of monocytes, especially when aggregated on rather large particles, e.g. 1.0 μm to 50 μm in diameter, particles carrying anti-CD14 receptor antibodies, will be withheld by such filters.

In a non-limiting sense, the said “first filter” may also consist of glass, glass fibre, polypropylene, polyethylene, fluoropolymer, cellulose, nitrocellulose, polyamide and blends thereof. In general, a blocking treatment against unspecific binding of proteins and cells is preferred. When T-lymphocytes is the said specific group of cells in a sample of whole blood or a sample derived from blood, a pore size of the filter of 18 to 50 μm is suitable, however a preferred pore size is 22 to 40 μm, and even more preferred is a pore size of 25-33 nm.

In a next step (“third step”), the method of the present invention comprises passing the remaining mixture through a “second filter” retaining the said “specific group” of cells in said sample, however letting receptor molecules in solution pass through the filter. The pore size of this “second filter” is, therefore, smaller than the pore size of the first filter.

In a non-limiting sense, the said “second filter” may consist of glass, glass fibre, polypropylene, polyethylene, fluoropolymer, cellulose, nylon, nitrocellulose, polyamide and blends thereof. In general, a blocking treatment against unspecific binding of proteins and cells is preferred. If the said “specific group” of cells is T-lymphocytes, a suitable pore size of said membrane for capturing the T-cells are membranes with an average pore size 1-10 μm, preferentially 3-9 μm, and even more preferred 5-8 μm, allowing smaller particulate materials from the sample materials after the hypotonic solution to pass through the membrane. If the said “specific group” of cells is constituted by other cells, other pore sizes are preferred.

The said “second filter” may then optionally be washed by a washing buffer or a washing solution.

In one embodiment of the invention, the said “specific group” of cells is constituted by the lymphocytes, including the T lymphocytes, often called the CD4+ T-cells.

In a next step (“fourth step”) of the method of the present invention, the said “second filter” is exposed to a liquid comprising labeled antibodies specifically reactive to said receptors, where said label is constituted by an enzyme or colored or fluorescent particle, optionally followed by a washing step.

### BRIEF DESCRIPTION OF THE DRAWINGS

FIG. 1 shows a vertical flow assay device which comprises an upper cover sheet (101) provided with at least one circular liquid sample feed opening (102) and a lower absorbent layer (105) fixed to said upper cover sheet (101); a first circular filter (106), removably inserted into said at least one circular opening (102); a second filter (104) fixed between said upper cover sheet (101) and said lower absorbent layer (105), and separating said at least one feed opening (102) and the circular filter (106) inserted therein from the absorbent layer (105).

FIG. 2 show a perspective view on another variant of a vertical flow assay device embodiment of the invention comprising an upper rotatable casing element (1) and a lower casing element (2) and a sample feed opening (3) and a reading opening (4).

FIG. 3 shows the device of FIG. 2 insertable into a corresponding opening (10a) provided in a card (10).

FIG. 4 shows a cross section of the assay device according to the FIG. 2.

FIG. 5 shows a top view of another embodiment of the assay device of the invention a sample provided with two pairs of feed openings and a reading openings (3, 4 and 3′, 4′).

### DETAILED DESCRIPTION OF THE INVENTION

### A. General Definitions

A “whole blood” sample as used in the assay method according to the invention is a sample derived from a mammal, in particular a human being. Any “Whole blood sample” may be used. Said samples may be used “as is”, i.e. without any pre-treatment, directly as taken from the blood donor, or may be pre-treated prior to the assay. Thus, for example whole blood in this context means a non-modified sample of whole blood or a sample where an anticoagulant has been added to the sample or a sample derived from whole blood, e.g. by adding a buffer or another liquid. Examples of suitable samples are native, untreated whole blood and pre-treated whole-blood blood, like EDTA blood, citrate blood, heparin blood. The originally obtained samples may be further modified by dilution. Fractionation of whole blood to remove constituents which might disturb the assay is normally not required. Dilution may be performed by mixing the original sample with a suitable sample liquid, like a suitable buffer, in order to adjust the concentration of the constituents, as for example of the analyte. The sample may also be pre-treated by hemolysis, as for example selective hemolysis of erythrocytes. Such modified samples exemplify samples “derived from” the original whole blood sample collected or isolated from the body of the mammal.

An “analyte” to be assayed according to the invention is a cell marker, in particular a cell surface marker, more particularly CD4 or CD8.

“CD4” (cluster of differentiation 4) is a glycoprotein found on the surface of immune cells such as T helper cells, monocytes, macrophages, and dendritic cells. It was discovered in the late 1970s and was originally known as leu-3 and T4 before being named CD4 in 1984.

“CD4+ T helper cells” are white blood cells that are an essential part of the human immune system. They are often referred to as CD4 cells, T-helper cells or T4 cells. They are called helper cells because one of their main roles is to send signals to other types of immune cells, including CD8 killer cells, which then destroy the infectious particle. If CD4 cells become depleted, for example in untreated HIV infection, or following immune suppression prior to a transplant, the body is left vulnerable to a wide range of infections that it would otherwise have been able to fight.

“CD8” (cluster of differentiation 8) is a transmembrane glycoprotein that serves as a coreceptor for the T cell receptor (TCR). Like the TCR, CD8 binds to a major histocompatibility complex (MHC) molecule, but is specific for the class I MHC protein. There are two isoforms of the protein, alpha and beta, each encoded by a different gene. The CD8 co-receptor is predominantly expressed on the surface of cytotoxic T cells, but can also be found on natural killer cells, cortical thymocytes, and dendritic cells.

“CD14” (cluster of differentiation 14), also known as CD14, is a human gene. The protein encoded by this gene is a component of the innate immune system. CD14 exists in two forms, one anchored to the membrane by a glycosylphosphatidylinositol tail (mCD14), the other a soluble form (sCD14). Soluble CD14 either appears after shedding of mCD14 (48 kDa) or is directly secreted from intracellular vesicles (56 kDa). CD14 is expressed mainly by macrophages and (at 10-times lesser extent) by neutrophils. It is also expressed by dendritic cells and monocytes.

A “Blood cell of interest” (BCol) as referred to herein belongs to a class or population or, more particular, to a sub-class or sub-population of cells typically present in a whole blood sample to be assessed according to the invention. Such (sub)-classes or (sub)populations are distinguishable from each other in the test environment (whole blood sample) on the basis of a particular cell surface marker or a pattern of such markers which may be analyzed by means of corresponding antibody molecules specific for said marker or pattern of markers.

A “sub-class”, “sub-set” or “sub-population” of cells refers to a group of blood cells which are functionally and antigenically related. Examples thereof are (CD4+) T-Helper cells or (CD8+) cytotoxic T cells.

Examples of a “class” or “population” of blood cells are T-lymphocytes and Blymphocytes.

“Distinguishable” in the context of the present invention means that the particular marker is either “specific” for said particular BCol, i.e. is not detectable in any other body cell; or is “subclass-specific” and therefore not detectable in another cell population of the blood sample to be analyzed; or is “non-specific” as it is detectable on other blood cells which are present in the whole blood sample as well, however, which are either present in a very low proportion, and does not negatively affect or falsify the assay result, or are removed from the sample before the assessment of the BCol is performed.

“Specific for” a class, population, sub-class or sub-population of cells in the context of the present invention, therefore, has to be understood broadly if not otherwise stated.

“Assessing” or “assessment” is intended to include both quantitative and qualitative determination in the sense of obtaining an absolute value for the amount or concentration of the analyte, present in the sample, and also obtaining an index, ratio, percentage, visual or other value indicative of the level of analyte in the sample. Assessment may be direct or indirect and the chemical species actually detected need not of course be the analyte itself but may for example be a derivative thereof.

The “accuracy” of an analytical method of the present invention, is the methods ability to accurately determine the concentration of the analyte in a sample, compared to the concentration as determined by an even more reliable reference method.

The “precision” of an analytical method of the present invention, is the variation in the results when the concentration of the analyte in a sample is determined repeatedly.

A “robustness” of an assay according to the present invention is the methods ability to tolerate interfering substances and variations in assay conditions without influencing the resulting value of the analyte concentration determination.

An “inert protein” as used in the context of the invention is a protein of any origin (for example, human or non-human mammalian, microbial) which does not disturb the assay method of the invention; in particular, it should have substantially no or no detectable affinity for the analyte to be analysed and/or for the antibodies as used in the assay method of the invention.

The term “particle size” is if not otherwise stated herein defined as “mean particle size”. Preferably the particles of the present invention, in particular the nanoparticles and immunoparticles derived therefrom by coupling of antibodies thereto, a characterized by a narrow, in particular an “essentially monomodal” or “monomodal” particle size distribution. Particle size determination may be performed in a manner known per se, as for example by applying particle size distribution measurements on a Malvern Mastersizer instrument. Typically, the measurement may be performed in 0.1M NaOH. The mean particle size values stated herein are either D(0.5) or D(4.3) values which may slightly differ but which, nevertheless are in the indicated parameter range, D(0.5) represents the mean particle size in μm at which 50% of the distribution is smaller and 50% of the size distribution is larger. D(4,3) represents the volume mean diameter. Mean particle sizes may also be determined microscopically, as for example by transmission electron microscopy (TEM).

“Antibody” relates to any class of “immunoglobulin molecule” (like IgA, D, E G, M, W, Y) and any isotype, including without limitation IgA1, IgA2, IgG1, IgG2, IgG3 and IgG4. Said term refers, in particular, to a functional (i.e. having the ability to bind to an antigen) monoclonal or polyclonal antibody (Ab) or fragment antibody (fAb) capable of binding to a particular antigen. Said Abs and fAbs are selected from chemically or enzymatically produced molecules or may be produced non-recombinantly or recombinantly by prokaryotic or eukaryotic microorganism or cell lines, or may be produced by higher organisms, like mammalian, preferably non-human mammalian species, or nonmammalian species, preferably avian species, or plants. Said fAbs may be selected from the group consisting of: monovalent antibodies (consisting of one heavy and one light chain), Fab, F(ab′)₂ (or Fab₂), Fab₃, scFv, bis-scFv, minibody, diabody, triabody, tetrabody, tandab; and single antibody domains, like V_{H} and V_{L} domains, and fragments thereof; wherein polyvalent fragments thereof may bind to different or, preferably, the same antigenic determinant of the same antigen, like in particular CD4 or CD8.

The term “labelled antibody” as used herein, refers to an antibody molecule as defined above with a label incorporated that provides for the identification of the antibody (preferably after binding to the respective antigen. Particularly, the label is a “detectable marker”, e.g., incorporation of a radio-labelled amino acid or attachment to a polypeptide of biotinyl moieties that can be detected by marked avidin (e.g., streptavidin containing a fluorescent marker or enzymatic activity that can be detected by optical or colorimetric methods). Examples of labels for antibodies include, but are not limited to, the following:
radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm);
fluorescent labels (e.g., FITC, rhodamine, lanthanide phosphors),
enzymatic labels (e.g., horseradish peroxidase, luciferase, alkaline phosphatase);
chemiluminescent markers;
biotinyl groups;
predetermined polypeptide epitopes recognized by a secondary reporter (e.g., leucine zipper pair sequences, binding sites for secondary antibodies, metal binding domains, epitope tags); and
polymer particles (e.g. colored nanoparticles)
metal particles (like gold nanoparticles)
magnetic agents, such as gadolinium chelates and
oligonucleotides.

The term “epitope” or “antigenic determinant” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to “specifically” bind an antigen when it at least preferentially or exclusively recognizes its target antigen in a complex mixture of proteins and/or macromolecules.

“Present on the surface” of a cell means that said molecule (like cell surface marker) is either bound to the cell surface or is integral part of the cell membrane and extends beyond the cell membrane into the extra-cellular space and optionally also into the intra-cellular space (i.e. the cytoplasm).

“Specific for” in the context of a reaction comprising the binding of a binding agent (like an antibody) to a target (like in particular an antigen, like CD4 or CD8), defines the ability of the binding agent to specifically recognize and bind said particular intended target while showing no cross-reactivity with a different target (in particular antigen) which might also be present in the sample to be analyzed.

“Haemolysed” or “Haemolysis” defines, that the red blood cells (RBCs) as contained in a whole blood sample do undergo a haemolytic cell disruption during, and preferably prior to the analytical assessment according to the present invention. Unless otherwise stated it refers in particular to the hypotonic lysis of erythrocytes without lysis of leucocytes (as for example described by Cunha et al in Anal. Methods, 2014, 6, 1377-1383, entitled “Kinetics of hypotonic lysis of human erythrocytes”)

“Agglutination” and “aggregation” (“agglutinate” and “aggregate”) are used as synonyms herein. These terms describe the clumping of particles. Agglutination occurs if an antigen is mixed with its corresponding antibody (also called isoagglutinin). The clumping of cells such as red blood cells in the presence of an antibody or complement or other molecules like lectins. The antibody or other molecule binds multiple particles and joins them, creating a large complex

A “vertical flow assay” or “vertical flow immune assay” according to the present invention is characterized by the vertical flow of a fluid through the assay device. The assay device comprises a multiplicity (i.e. at least two or more particularly three) layers either of identical or, preferably, of different functionality, as for example with respect to selective permeability (size exclusion) or different absorption characteristics for liquids, stacked one upon the other. Such functional layers may be selected from grids, filter membranes and adsorbent layers.

An “absorbent layer” comprises a suitable natural or synthetic material which has the ability to physically absorb the liquid phase (including constituents dissolved or suspended therein) of the sample to be analyzed, the washing liquids added during the assay method as well as the liquid phase of the liquid reagent medium (solution or dispersion of required reagents in a liquid phase) added into the device as well as unreacted constituents of said reagent medium. The size (volume) of said absorbent layer depends on the total volume of liquid to be absorbed and the absorption capacity of the absorbent material and should preferably exceed the volume of the liquid to be absorbed.

Unless otherwise stated the term “upper” refers to the side of the device at which the sample to be analyzed (as for example an optionally pre-treated blood sample) is added and enters the device.

Unless otherwise stated the term “inner” refers to those parts of the device which are not or substantially not in direct contact with the surrounding environment.

The “first configuration” of a device may also be designated as “sample addition configuration”.

The “second configuration” of a device may also be designated as “reagent addition configuration” or “read-out configuration” or “reading configuration”.

The “first opening” of a device may also be designated as “sample addition opening” or “sample feed opening”. In said opening the optionally pre-treated blood sample is added and washed into the first filter layer, so that cell agglomerates optionally formed in said sample are retained by said filter.

The “second opening” of a device may also be designated as “reagent addition opening”, “reading opening” or “read-out opening”. A detectable signal formed upon addition of a reagent specific for the analyte (as for example cells or cell surface markers to be assessed) may be detected and read out from said opening.

“Multiplex” detection relates to the simultaneous detection of different analytes (like antigens) in the same sample, and preferably in the same assay device, at the same or different spots.

Multiplexing is easily achieved by spotting the same sample at one or more predetermined locations and/or patterns on the assay device. For easier visualization multiplexing can also be coupled with analytical probes (as for example antibodies) carrying distinguishable labels, as for example coupled to nanoparticles of different color. If different spots are applied for different analytes the presence of a particular antigen is easily detectable by the appearance of the corresponding label (color) signal. If one single spot is applied the a mixed label (color) will appear if two or more different antigens are present in the sample, and the composition of the mixed label (color) will have to be analyzed in a suitable manner, as for example, spectroscopically.

### B. Preferred Embodiments

B1. The present invention relates to the following particular embodiments:
1. An assay method for assessing in a liquid whole blood sample or a sample derived therefrom, one or more sub-classes of blood cells of interest (BCol), each sub-class carrying a first, preferably distinguishable, cell surface marker (or cell surface receptor molecule) (M1) for said sub-class of blood cells of interest, which means that the markers (M1) for different sub-classes of cells are different (i.e. antigenically different and therefore distinguishable) from each other, wherein said sample may additionally comprise (or is suspected to comprise) disturbing blood cells (DBC), which carry at least one of said first cell surface markers (M1) as non-specific marker and would thus disturb the assessment of the said subclass of BCol also carrying at least one of said markers (M1), and/or wherein said sample may additionally comprise (or is suspected to comprise) at least one free (as for example dissolved), non-cell surface bound form, like a (for example soluble) extracellular fragment, of at least one, preferably of each of said first cell surface markers (M1), which method comprises
   (1) removing from said sample any disturbing blood cells (DBC), which also carry at least one of said first cell surface markers (M1);
   (2) removing from said sample as obtained in step (1) any free, non-cell surface bound form of each of said first cell surface markers (M1); and
   (3) assessing in the sample as obtained in step (2) each of said sub-classes of BCol, carrying said first cell surface marker (M1).
   In particular, said whole blood sample is blood from a mammalian, preferably human, individual, like a blood donor, or a patient suffering from a disease or suspected to suffer from a disease affecting the cellular profile or composition of the population of whole blood cells, in particular of at least one of said BCol. It can be obtained e.g. from venous collection through a needle, or from capillary blood collected after a finger stick by a sharp object.
   In a first particular alternative the present method comprises the assessment of one single sub-class of BCol, and steps (1) to (3) are performed once. Preferably, said one single sub-class comprises CD4⁺ cells, and the surface marker M1 is CD4. The DBC comprise CD14⁺ cells which also carry the M1 marker CD4, in particular said DBC comprise CD14⁺ monocytes. Said non-cell surface bound form of said first cell surface marker M1 is derived from CD4, i.e. comprises a soluble fragment thereof.
   In a second particular alternative the present method comprises the multiplex assessment of two different sub-classes of BCol and steps (1) to (3) are performed separately for each subclass of cells.
   In a variant of said second particular alternative the present method comprises the multiplex assessment of two different sub-classes of BCol (as for example CD4+ cells and CD8+ cells) and steps (1) to (3) are performed for a first subclass of BCol (as for example CD4+ cells) and at least steps (2) and (3) are separately performed for the second sub-class of cells (as for example CD8+ cells) if no other blood cells would disturb the assessment of said second sub-class of cells.
   Preferably, said two different sub-classes comprises CD4⁺ cells (the first subclass) and CD8⁺ cells (the second subclass) and the surface markers M1 to be assessed are CD4 (i.e. M1a) and CD8 (i.e. M1b). The DBC comprise CD14⁺ cells, in particular CD14⁺ monocytes, which also carry said CD4 marker (M1a). Said non-cell surface bound form of said markers M1a and M1b is derived from CD4 and/or CD8, i.e. comprises a soluble, non-cell bound fragment of CD4 and/or CD8.
   In a third particular alternative the present method comprises the multiplex assessment of two different sub-classes of BCol and steps (1) to (3) are performed only once.
   In a fourth particular alternative the present method comprises the multiplex assessment of two different subclasses of BCol and steps (1) and (2) are performed only once while step (3) is performed for each of said subclasses separately.
   Preferably in said above second, third and fourth alternatives, said two different sub-classes comprises CD4⁺ cells (the first sub-class) and CD8⁺ cells (the second subclass) and the surface markers M1 to be assessed are CD4 (i.e. M1a) and CD8 (i.e. M1b). The DBC comprise CD14⁺ cells, in particular CD14⁺ monocytes, which also carry said CD4 marker (M1a). Said non-cell surface bound form of said markers M1a and M1b is derived from CD4 and/or CD8, i.e. comprises a soluble fragment of CD4 and/or CD8.
2. The assay method of embodiment 1, which is a vertical flow assay method, in particular a vertical flow immunoassay.
3. The assay method of one of the preceding embodiments, wherein in step (1) said DBCs are removed by filtration, in particular through a grid or net, as for example a Nylon net.
4. The assay method of embodiment 3, wherein said DBCs are aggregated, which aggregates are retained by the filter applied in step (1).
5. The method of embodiment 4, wherein said DBCs are aggregated by means of immunoglobulin molecules which do not bind said BCol.
6. The method of embodiment 5, wherein said DBCs are aggregated by means of immunoglobulin molecules, which bind to a second cell surface marker (M2) which is not present on the surface of said BCol (and thus may be identified as distinguishable marker), in particular wherein said second cell surface marker (M2) is distinguishable for, or may even be specific for said DBCs.
7. The method of embodiment 5 or 6, wherein said DBC binding immunoglobulins are selected from free antibodies, polymeric antibodies or antibodies, bound to the surface of solid particles, in particular polymer particles.
8. The method of one of the preceding embodiments, wherein in step (2) said non-cell surface bound form of said first cell surface marker (M1) is removed by filtration by applying a filter which is permeable for said non-cell surface bound form of said first cell surface marker (M1) but which retains said BCol.
9. The method of one of the preceding embodiments, wherein said assessment of step (3) is performed by means of immunoglobulin molecules, preferably monoclonal or polyclonal, non-human, like rodent or avian antibodies, (specifically) reactive with said first cell surface marker (M1), preferably an extracellular part of said marker.
10. The method of embodiment 9, wherein said immunoglobulin molecules are labelled.
11. The method of embodiment 10, wherein said label is selected from an enzyme, a fluorescent or colored molecular marker or a fluorescent or colored particle.
12. The method of one of the preceding embodiments, wherein said BCol are selected from a sub-class of lymphocytes, in particular T-lymphocytes, and said DBCs are monocytes.
13. The method of one of the preceding embodiments, wherein said first cell surface marker (M1) is a T-lymphocyte marker (M1a), in particular the CD4 cell surface receptor molecule.
14. The method of one of the preceding embodiments, wherein said one or more sub-classes of blood cells of interest (BCol) to be assessed comprises CD4⁺ cells.
15. The method of one of the preceding embodiments, where said first cell surface marker (M1a) is CD4 and said specific first sub-class of cells is T-helper cells.
16. The method of one of the preceding embodiments, where said method also comprises the assessment of a second sub-class of blood cells (BCol) carrying a second, preferably distinguishable, cell surface marker (M1b) different from said first cell surface marker (M1a).
17. The method of embodiment 16, wherein said cell surface marker (M1b) is a T-lymphocyte marker different from (M1a), in particular the surface marker CD8 and said specific second sub-class of cells comprises CD8⁺ cells.
18. The method of embodiment 17, where said surface marker (M1b) is CD8 and said specific second sub-class of cells is cytotoxic T-cells.
19. The method of one of the embodiments 16 to 18, wherein the assessment of said second sub-class of BCol carrying said second cell surface marker (M1b) is performed in step (3) together with the assessment of said first subclass of BCol, carrying a first cell surface marker (M1a), in particular in the same sample.
20. The method of one of the embodiments 16 to 18, wherein the assessment of said second sub-class of BCol carrying said marker (M1b) is performed separately.
21. The method of embodiment 20, which method comprises
   (4) optionally removing from said sample any disturbing macromolecular impurities which might disturb the assessment;
   (5) removing from said sample (optionally as obtained in step (4)) any free, non-cell surface bound form of said first cell surface markers (M1b); and
(6) assessing in the sample as obtained in step (5) said sub-class of BCol carrying said cell surface marker (M1b).
22. The method of embodiment 21, wherein in step (5) said non-cell surface bound form of said second cell surface marker (M1b) is removed by filtration by applying a filter which is permeable for said non-cell surface bound form of said cell surface marker (M1b) but which retains said sub-class of BCol carrying (M1b).
23. The method of embodiment 22, wherein said assessment of step (6) is performed by means of immunoglobulin molecules, preferably monoclonal or polyclonal, non-human, like rodent or avian antibodies, reactive with said cell surface marker (M1b), preferably an extracellular part of said marker.
24. The method of embodiment 23, wherein said immunoglobulin molecules are labelled.
25. The method of embodiment 24, wherein said label is selected from an enzyme, a fluorescent or colored molecular marker or a fluorescent or colored particle.
26. The method of one of the preceding embodiments, wherein said DBCs are CD14⁺ monocytes.
27. The method one of the preceding embodiments, where the aggregation of DBCs in step (1) is performed by adding a first liquid comprising immunoglobulins, preferably monoclonal or polyclonal, non-human, like rodent or avian antibodies, said liquid being able to lyse erythrocytes contained in the sample.
28. The method one of the preceding embodiments, comprising the steps of
   (1a) mixing the said sample or an aliquot of the said sample with a first liquid comprising antibodies binding to other structures on the surface of other cells (in particular DBC) different from said specific sub-group of cells (in particular CD4⁺ cells) but carrying said CD4 receptors, forming particles or aggregates or clusters of particles or cells with a size significantly larger than the size of the cells in said specific sub-group of cells,
   (1b) filter away said formed particles or aggregates or cluster of particles or cells by means of a first filter that is constituted by a size exclusion filter, and
   (2) passing the remaining mixture through a second filter retaining the said specific sub-group of cells (in particular CD4⁺-cells) in said sample but letting CD4 receptor molecules in solution pass through the filter, optionally followed by a washing step,
   (3a) followed by exposing the said second filter to a liquid comprising labeled antibodies specifically reactive to said CD4 receptors, where said label is constituted by an enzyme or colored or fluorescent particle, optionally followed by a washing step,
   (3b) optionally followed by adding a substrate to said enzyme generating a colored or fluorescent substance, and
   (3c) measuring the intensity of the color or the fluorescence on said second filter and correlating said intensity to the concentration of said class of CD4 receptors on the surface of the said specific sub-group of cells (in particular CD4⁺-cells).
29. The method of one of the preceding embodiments, wherein a (selective) hypotonic lysis of erythrocytes is performed to said blood sample prior to the assessment (i.e. before step (1) is performed, preferably a hypotonic lysis of erythrocytes without lysis of leucocytes (as for example described by Cunha et al in Anal. Methods, 2014, 6, 1377-1383, entitled “Kinetics of hypotonic lysis of human erythrocytes”) is performed.
30. The method of one of the preceding embodiments, wherein the cell count for the group of CD4⁺ cells is assessed (in number of cells/volume of sample).
31. The method of embodiment 30, wherein the cell count for the group of CD4⁺ cells, and at least for one further group of cells, different from CD4⁺ cells, in particular for the group of CD8⁺ cells, is assessed, in particular the CD4/CD8 ratio.
32. A method for assessing the quantity of CD4 receptors located on the surfaces of CD4⁺ cells and optionally for assessing the quantity of CD8 receptors located on the surfaces of CD8⁺ cells in a sample of whole blood or a sample derived from blood, which method comprises performing a method of one of the embodiments 1 to 29 and correlating the signal obtained for the assessment of the group of CD4⁺ cells with the quantity of cell-bound CD4⁺ receptor, and optionally correlating the signal obtained for the assessment of the group of CD8⁺ cells with the quantity of cell-bound CD8⁺ receptor.
33. The method of one of the preceding embodiments, wherein said immunoglobulin molecules as applied in said method are antibodies, like monoclonal or polyclonal non-human, in particular non-rodent antibodies, like avian (in particular anti CD4, anti CD8 and anti CD14 antibodies).
34. The method of one of the preceding embodiments, wherein the immunoglobulins applied for binding to (M1a) and/or (M1b), in particular to CD4⁺ or CD8⁺ cells, are covalently bound to colored latex particles having a mean particle diameter (before being coated with said immunoglobulins) in the range of 30 to 500 nm.
35. A vertical flow assay device for performing the method of any of the embodiments 1 to 34, which device comprises
   an upper cover sheet (101) provided with at least one circular, preferably liquid, sample feed opening (102) and a lower absorbent layer (105) fixed to said upper cover sheet (1);
   a first circular filter (106), removably inserted into said at least one circular opening (102);
   a second filter (104) fixed between said upper cover sheet (101) and said lower absorbent layer (105), and separating said at least one feed opening (102) and the circular filter (106) inserted therein from the absorbent layer (105).
36. The device of embodiment 35, wherein said first circular filter (106) is fixed via a carrier ring (108) to an adhesive tape (107), said ring (108) having an outer diameter slightly smaller than the diameter of the sample feed opening (102), and having an inner diameter chosen to define a free circular space sufficient for quantitatively taking up a predetermined sample volume.
37. The device of embodiment 36, wherein said tape (107) removably adheres to the upper side of said an upper cover sheet (101).
38. The device of embodiment 35 to 37, wherein said a first circular filter (106), removably inserted intro said at least one circular opening (102), is removed from the device by removing the tape (107) from said upper cover sheet (101).
39. An assay device, comprising
   an upper casing element (1) and a lower casing element (2),
   the upper (1) and the lower casing element (2) being assembled in such a manner that a testing compartment is formed, which is suited to take up a stack of functional layers (5, 6, 7),
   the testing compartment comprising an upper testing compartment inner surface (1a) of the upper casing element (1) and a lower testing compartment inner surface (2a) of the lower casing element (2),
   the upper casing element (1) being movable with respect to the lower casing element (2), thereby defining a first configuration and a second configuration of the assay device,
   the upper casing element (1) having a first opening (3) and a second opening (4), which both provide access from the outside to the testing compartment,
   the first opening (3) and the second opening (4) being arranged in such a manner that the position of the first opening (3) with respect to the lower casing element (2) at the first configuration is essentially the same as the position of the second opening (4) with respect to the lower casing element (2) at the second configuration.
40. The assay device according to embodiment 39, characterized in that the upper casing element (1) is rotatable with respect to the lower casing element (2).
41. Assay device according to one of the embodiments 39 and 40, characterized in that the stack of functional layers comprises an upper membrane layer (6) and a lower absorbent layer (7), which are arranged on top of each other and extend essentially in parallel to the upper testing compartment surface (1a) and the lower testing compartment surface (2a).
42. Assay device according to one of the preceding embodiments 39 to 41, characterized in that at least the upper membrane layer (6) is fixed to the lower casing element (2).
43. Assay device according to one of the embodiments 39 to 42, characterized in that a movement limiter (21) is formed in the upper casing element (1) and another movement limiter (22) is formed in the lower casing element (2), wherein the movement limiters (21,22) are provided in such a manner that the upper casing element (1) is movable with respect to the lower casing element (2) between a first extreme position corresponding to the first configuration and a second extreme position corresponding to the second configuration.
44. Assay device according to one of the embodiments 39 to 43, characterized in that the upper casing element (1) has several first openings (3, 3′) and second openings (4,4′), every one of the first openings (3,3′) being associated with one second opening (4,4′), wherein the first openings (3,3′) and the second openings (4,4′) are arranged in such a manner that the positions of the first openings (3,3′) with respect to the lower casing element (2) at the first configuration are essentially the same as the position of the associated second openings (4,4′) with respect to the lower casing element (2) at the second configuration.
45. The device of one of the embodiments 35 to 44, wherein said filter (106, 5) has openings or pores retaining aggregated blood cells, in particular, aggregated CD14⁺ monocytes, and is permeable for non-aggregated blood cells, in particular CD4⁺ cells and optionally CD8⁺ cells.
46. The device of embodiment 45, wherein said filter (106, 5) is a net filter having a grid size in the range of 18 to 50 μm, preferably 22 to 40 μm, more preferably 25 to 33 μm.
47. The device of one of the embodiments 35 to 46, wherein said second filter (104) or membrane element (6) has openings or pores retaining non-aggregated blood cells and is permeable to constituents soluble in said liquid sample.
48. The device of embodiment 47, wherein said second filter (104) or membrane element (6) has a pore size in the range of 1 to 10 μm, preferably 3 to 9 μm, more preferably 5 to 8 μm.
49. The device of any of the embodiments 35 to 48, wherein said absorbent layer 105, 7) has an absorbing capacity sufficiently high to absorb any liquid constituents of sample and reagents and washing solutions added to the sample feed opening (102, 3, 3′) during the course of the vertical flow assay.
50. The use of a device as defined in anyone of the embodiments 35 to 49 for analytical or diagnostic purposes, in particular in medical diagnostics or analytics, and preferably for performing an assay as defined in anyone of the embodiments 1 to 34.

B2. Additional preferred embodiments are

In the context of the following embodiments the “class of receptors” refers in particular to the “CD4 receptor”

The “specific group of cells” refers in particular to “CD4+ cells”
I. A method for assessing the quantity of a class of receptors located on the surfaces of a specific group of cells in a sample of whole blood or a sample derived from blood, characterized by
   a) mixing the said sample or an aliquot of the said sample with a first liquid comprising antibodies binding to other structures on the surface of other cells different from said specific group of cells but carrying said receptors, forming particles or aggregates or clusters of particles or cells with a size significantly larger than the size of the cells in said specific group of cells,
   b) filter away said formed particles or aggregates or cluster of particles or cells by means of a first filter that is constituted by a size exclusion filter, and
   c) passing the remaining mixture through a second filter retaining the said specific group of cells in said sample but letting CD4 receptor molecules in solution pass through the filter, optionally followed by a washing step,
   d) followed by exposing the said second filter to a liquid comprising labeled antibodies specifically reactive to said receptors, where said label is constituted by an enzyme or colored or fluorescent particle, optionally followed by a washing step,
   e) optionally followed by adding a substrate to said enzyme generating a colored or fluorescent substance, and
   f) measuring the intensity of the color or the fluorescence on said second filter and correlating said intensity to the concentration of said class of receptors on the surface of the said specific group of cells.
II. The method according to embodiment I for assessing the quantity of a class of receptors located on the surfaces of a specific group of cells in a sample of whole blood or a sample derived from blood, characterized by
   a) mixing the said sample or an aliquot of the said sample with a first liquid comprising antibodies binding to other structures on the surface of other cells different from said specific group of cells but carrying said receptors, forming particles or aggregates or clusters of particles or cells with a size significantly larger than the size of the cells in said specific group of cells,
   b) filter away said formed particles or aggregates or cluster of particles or cells by means of a first filter that is constituted by a size exclusion filter, and
   c) passing the remaining mixture through a second filter retaining the said specific group of cells in said sample but letting CD4 receptor molecules in solution pass through the filter, optionally followed by a washing step,
   d) followed by exposing the said second filter to a liquid comprising labeled antibodies specifically reactive to said receptors, where said label is constituted by a colored or fluorescent particle, optionally followed by a washing step, and
   e) measuring the intensity of the color or the fluorescence on said second filter and correlating said intensity to the concentration of said class of receptors on the surface of the said specific group of cells.
III. The method according to anyone of the embodiment I or II, where said receptor is CD4 and said specific group of cells is T lymphocytes.
IV. The method according to any of the embodiments I to III, characterized by the antibodies binding to other structures on the surface of other cells different from said specific group of cells but carrying said receptors are antibodies reactive to receptor CD 14.
V. The method according to anyone of the embodiment I to IV, where said antibodies binding to other structures on the surface of other cells different from said specific group of cells but carrying said receptors, are polymerized antibodies or are immobilized on particles or polymers or other large molecules facilitating the formation of particles or aggregates or clusters of particles or cells with a size significantly larger than the size of the cells in said specific group of cells.
VI. The method according to anyone of the embodiment I to V, where the said first liquid comprising antibodies is a liquid that is able to lyse the erythrocytes of the sample.
VII. The method according to anyone of the embodiment I to VI, where the said first liquid comprising antibodies comprises antibodies with a specific reactivity to CD14 receptor molecules.

### C. Further Embodiments

C.1 CD4, CD8 or CD14—Binding Immunoglobulins

If not otherwise stated herein, such immunoglobulins are preferably directed against an extracellular part (antigen binding domain) of one of said markers. If isoforms of one of said markers exist said immunoglobulins may be directed to individual or all isoforms to be found in/on the DBCs to be removed or BCols to be assessed according to the invention.

C.1.1 Polyclonal Antibodies

Polyclonal anti-human CD4, CD8 or CD14 antibodies can be prepared by methods well known in the art, such as those described for example by Chase, M. W., 1967, in “Methods of Immunology and Immunochemistry”, ed. Williams, A. et al., M. W., pp. 197-209, Academic Press, New York. Briefly, animals of a suitable species (for example rabbits, goats, or sheep, or, preferably avian species, in particular poultry, like hens) are repetitively immunized with purified antigen in an appropriate adjuvant, for example Freund's complete or incomplete adjuvant. After immunization the animals are bled and the polyclonal antibodies are purified by methods such as for example ammonium sulfate or ammonium chloride precipitation, anionic exchange chromatography, immunoaffinity chromatography, and/or affinity chromatography.

To achieve very good signal, antibodies of high avidity may be preferred. Since polyclonal antibodies comprise many different antibody molecules, an affinity constant cannot be calculated, however high avidity and affinity was obtained by conventional polyclonal antibody techniques. Rabbit antibodies obtained by conventional methods were used, however even better results were obtained with sheep antibodies. Even more better results were obtained when avian antibodies were used. The avian antibodies may be according to the methods described in Larsson A, Baaloew R-M, Lindahl T, and Forsberg P-O in Poultry Science 72:1807-1812, 1993. It is contemplated that the avians being genetically more distinct from humans are able to generate antibodies towards human CD4, CD8 or CD14 that have a higher avidity than polyclonal mammalian antibodies.

Polyclonal avian antibodies routinely are obtained from egg yolk (and are therefore designated IgYs). Egg yolk, however, contains large amounts of lipids making their further use problematic. IgY can be isolated from egg yolk by using stepwise ammonium sulphate (for example 25 to 40%) and polyethylene glycol (PEG) precipitation. For initial purification also commercial IgY purification kits obtainable from Gallus Immunotch Inc, Cary, USA, or the Eggcellent Chicken IgY Purification Kit, obtainable from Pierce, Rockford, USA may also be employed considering the manufacturer's instructions.

Furthermore, the avidity of polyclonal antibodies may be further increased by using antibodies that were purified by the use of antigen affinity purification methods, for example according to the teaching in “Affinity Purification of Proteins” downloaded from www.piercenet.com (April 2006) and incorporated by reference Affinity purification is described in more detail below.

“Increased avidity” was in particular observed when 20% of the antibodies used had been antigen affinity purified, even more increase was observed when 50% of the antibodies had been antigen affinity purified and even more when more than 75%, like 75 to 100% of the antibodies had been obtained by antigen affinity purification methods.

For affinity purification of (for example avian) polyclonal anti-human CD4, CD8 or CD14 antibodies a suitable human CD4, CD8 or CD14 affinity column has to be prepared. Purified human CD4, CD8 or CD14 is fixed by a standard protocol to a suitable solid supports as for example are Sepharose or Affi-Gel, activated to covalently the antigen to the support (suitable activated solid supports are for example available from Pierce, Rockford, USA). An affinity column is then prepared from said antigen-carrying resin.

Successful affinity purification of antibody depends on effective presentation of the relevant epitopes on the antigen to binding sites of the antibody. If the antigen is small and immobilized directly to a solid support surface by multiple chemical bonds, important epitopes may be blocked or sterically hindered, prohibiting effective antibody binding. Therefore, it is best to immobilize antigens using a unique functional group (e.g., sulfhydryl on a single terminal cysteine in a peptide) and to use an activated support whose reactive groups occur on spacer arms that are several atoms long. For larger antigens, especially those with multiple sites of immobilization, the spacer arm length becomes less important since the antigen itself serves as an effective spacer between the support matrix and the epitope.

Little variation normally exists among typical binding and elution conditions for affinity purification of antibodies because at the core of each procedure is the affinity of an antibody for its respective antigen. Since antibodies are designed to recognize and bind antigens tightly under physiologic conditions, most affinity purification procedures use binding conditions that mimic physiologic pH and ionic strength. The most common binding buffers are phosphate buffered saline (PBS) and Tris buffered saline (TBS) at pH 7.2 and 1.5 M NaCl (premixed buffer packs are for example available from Pierce, Rockford, USA). Once the antibody has been bound to an immobilized antigen, additional binding buffer is used to wash unbound material from the support. To minimize non-specific binding, the wash buffer may contain additional salt or detergent to disrupt any weak interactions.

Specific, purified antibodies are eluted from an affinity resin by altering the pH and/or ionic strength of the buffer (common elution buffers are for example available from Pierce, Rockford, USA). Antibodies in general are resilient proteins that tolerate a range of pH from 2.5 to 11.5 with minimal loss of activity, and this is by far the most common elution strategy. In some cases, an antibody-antigen interaction is not efficiently disrupted by pH changes or is damaged by the pH, requiring that an alternate strategy be employed.

An example for an affinity purification protocol is given below:

Step 1:

Wash the column (˜1 ml resin bed) to remove residual protein before each use using 10 column volumes of the following sequence of buffers:
1. 0.2 M glycine, pH 2.8-10 ml
2. 0.1 M PBS, pH 7.2, 0.15 M NaCl ˜10 ml
3. Repeat the cycle with the above buffers twice. Then, equilibrate the column in the same PBS buffer with ˜5 ml

Step 2:

Centrifuge 10 ml of crude antibody preparation to remove precipitates.

Step 3:

Apply the crude antibody preparation to the column using a slow flow rate.

Step 4:

Wash the column extensively with 10 ml of 0.1 M PBS, pH 7.2, 0.15 M NaCl

Step 5:

Elute the antibody using 3 ml 0.15 M Ammonium Hydroxide, pH 10.5±0.2. Collect fractions into adequate tubes. Read the A₂₈₀ of each fraction using an appropriate blank (i.e., 0.15 M Ammonium Hydroxide, pH 10.5±0.2).

Step 6:

Pool the appropriate fractions. Get an A₂₈₀ of the pools and let the antibodies maturate at room temperature for a maximum of 2 weeks. If the antibodies are to be use immediately after maturation, follow the coating procedure. If contrary, the antibodies should be dialyzed against PBS containing a preservative, such as NaN₃ or Proclin 950, and stored at 4° C.

Step 7:

At the end, the column must be washed extensively with PBS containing a preservative, such as NaN₃ or Proclin 950.

C.1.2 Monoclonal Antibodies

Polyclonal antibodies are often more preferred than monoclonal antibodies in particleenhanced assays. Polyclonal antibodies, contrary to monoclonals, are inherently reactive to many different epitopes on the antigens (or analytes), and therefore more easily create cross-bindings and networks between the antigens molecules per se, and between the antigens and the particles to which the antibodies are immobilized. In contrast, monoclonal antibodies generally bind to one type of epitopes only, which makes it more difficult to form cross-bindings and networks. The diagnostic industry often prefers, however, the use of monoclonal antibodies, because they are easier to standardized and to quality control to a predefined standard, especially over a product life-time of many years. Cocktails of different monoclonal antibodies, especially when they are composed of many different monoclonal antibodies with high affinity to CD4, CD8 or CD14, will result in good embodiments of the present invention.

Monoclonal anti-human CD4, CD8 or CD14 antibodies also can be prepared by methods well known in the art, as for example those described by G. Köhler at al., 1975, Nature 256, 495, G. Galfre et al., 1981, Meth. Enzymol. 73, 3-46, or R. Kennet, 1980, in: “Hybridomas: a new dimension in biological analysis”, ed. R. Kennet et al., Plenum press, New York & London. Spleen cells or peripheral blood cells from immunized mice or rats are fused with a myeloma cell line, using for instance the polyethylene fusion method. After fusion the cells are grown under suitable conditions, for example on culture plates and a selection of correctly fused cells is performed using for example the hypoxanthine/aminopterin/thymidine (HAT) selection method. Antibody producing cell lines are identified by methods such as EIAs, RIAs or agglutination assays. After identification of the antibody producing cell line, the cells are repeatedly sub-cloned, as for example by the method of limited dilution, to guarantee that the new growing cell line derives from one single cell.

C.1.3 Chimeric Antibodies

Chimeric anti-human CD4, CD8 or CD14 antibodies can be obtained by methods well known in the art such as that described by G. L. Boulianne et al., 1984, Nature 312, 643-645. The procedure can be briefly described as follows. The DNA of the antigen-binding site from a monoclonal antibody of one species or parts thereof are transferred to the DNA of the antibody framework of another antibody of a different species. This new construct is cloned into an expression vector, which is transferred to the corresponding expression system to produce the antibody.

C.1.4 Recombinant Antibodies

Recombinant anti-human CD4, CD8 or CD14 antibodies can be obtained without using animal vehicles by methods known in the art, such as those described by G. Winter et al., 1991, Nature, 349, 293 or J. S. Huston et al., 1988, Proc. Ntl. Acad. Sci. USA, 85, 5879. Those methods involve the following steps: introduction of DNA (cDNA or synthetic DNA) coding for an antibody or fragments thereof into a host cell, for example E. coli, fungi, yeast, plants or eukaryotic cells, selection of antibodies with the desired specificity and affinity and expressing the antibody or fragment thereof in the corresponding expression system.

C.1.5 Antibody Fragments (fAbs)

Fragments as described herein above, like Fab-, and F(ab′)₂-fragments of polyclonal antibodies, monoclonal antibodies of any species (including chimeric antibodies and or recombinant antibodies) can be prepared by methods well known in the art, such as those described for example by A. Nissonoff et al., 1960, Arch Biochem Biophys, 89, 230, or R. P. Porter, 1959, Biochem J, 73, 119, or E. Harlow et al, 1988, in “Antibodies-A Laboratory Manual”, 626-631, Cold Spring Harbour Press, New York, USA.

C.1.6 Selection of Anti-Human CD4, CD8 or CD14 Antibodies of Different Reactivity to the Human CD4, CD8 or CD14

When using monoclonal antibodies or fragments thereof as binding partners, the selection of the antibodies of different, in particular high and low reactivity, can conveniently be performed by coating each of the monoclonal antibody separately onto nanoparticles of the same material and size by conventional coating techniques, followed by mixing the nanoparticle reagents in a given ratio, for example 1/1 v/v, in a permutative manner with the analyte. After generating calibration curves of the nanoparticle reagent under the same conditions, the steepness of the resulting calibration curves for low concentrations of analyte gives a first indication of the reactivity of the immunological binding partners.

When using polyclonal antibodies as binding partners, the preparation of high and low reactivity polyclonal antibodies may be performed according to methods well known in the art by introducing the polyclonal antibodies into an affinity chromatography column, carrying the antigenic analyte covalently bound to the gel matrix. With a gradient of elution buffer low reactivity polyclonal antibody fractions will elute first from the column, followed by fractions with increasingly higher reactivity (see S. Yamamoto et al., 1993, “Veterinary Immunology and Immunopathology” 36, 257-264, Elsevier Science Publishers B.V., Amsterdam). Reactivity of the fractions can then be checked either with a BIAcore instrument or by coating them independently onto nanoparticles of the same size and material and generating the corresponding calibration curves.

Selection of antibodies can be done by the above mentioned procedure of coating them on nanoparticles followed by a detection limit analysis or a determination of its functional affinity as described above. If appropriate, mixtures of different anti-human CD4, CD8 or CD14 antibodies differing with respect to their affinity/avidity vis-à-vis human CD4, CD8 or CD14 may be used for preparing nanoparticle-antibody conjugates of the present invention. Suitable mixing ratios can be determined by a skilled person by a limited series of experiments.

Suitable anti-human CD4, CD8 or CD14 antibodies are also commercially available from different sources. (see also experimental section).

C1.7 Polymeric Antibodies

The preparation of polymeric multifunctional antibodies is well known in the art. A suitable method is for example described in EP-A-0 957 363.

C.3 Nanoparticles (latex particles) and their conjugates with antibody

Such nanoparticles are either applied in the step of agglutinating DBCs are applied for the detection of the cell surface markers M1.

The material for preparing the nanoparticles as used in the invention may be any natural or synthetic, inorganic, organic, non-polymer or polymer material suitable for generating and performing particle-enhanced light scattering assays. Such materials include for example selenium, carbon, gold; nitrides of carbon, silicium or germanium, for example Si₃N₄; oxides of iron, titanium or silicium, for example TiO₂ or SiO₂; and polymeric materials such as for example, polystyrene, poly(vinyl chloride), epoxy resins, poly(vinylidene chloride), poly(alpha-naphtyl methacrylate), poly(vinylnaphthalene), or copolymers thereof, in particular copolymers of styrene and a copolymerizable ethylenically unsaturated compound, for example styrene-(meth)acrylate co-polymers. Particles made of polymeric materials, as well as core-shell particles consisting of an inner core polymerized from styrene and an outer shell formed by copolymerization from styrene with a copolymerizable, ethylenically unsaturated compound, as described for example in U.S. Pat. No. 4,210,723, are also suitable.

Suitable polymeric particles for conjugation can be purchased from Bangs Particles Inc. or Interfacial Dynamics Inc, Merck SA, France, or other suitable sources. The particles can be activated for binding to antibodies according to numerous methods, a thorough teaching of such coupling chemistry can be found, e.g. in TechNote 205, Rev. 003, for example March, 2002, “Covalent Coupling” (incorporated by reference) which can be downloaded from Bangs Laboratories, Inc.'s web-site. For example, coupling may be achieved by means of particles carrying on their surface carboxyl-, amino-, hydroxyl-, hydrazide- or chloromethyl groups. The molecule to be coupled may either react directly with such groups or by means of a suitable linker, as for example carbodiimides, glutaraldehyde, or cyanogen bromide.

For detection purposes of markers (M1) the nanoparticles (conjugated with a suitable anti M1-antibody may be further modified by the attachment of a detectable marker, like a fluorophore or a chromophore. Corresponding particles are commercially available or may be produces by suitable preparative methods well known in the art (se for example: Site-specific labelling of proteins using cyanine dye reporters described in CA 2493309 A1; Protein specific fluorescent microspheres for labelling a protein described in U.S. Pat. No. 4,326,008 A; or Protein specific fluorescent microspheres for labelling a protein described in U.S. Pat. No. 4,326,008 A).

C.4 Performing the Method of the Invention and Equipment Therefor

C.4.1 Devices

Suitable devices are also disclosed in co-pending EP application, application number EP16180938.9 by Gentian AS, which document is herewith incorporated by reference.

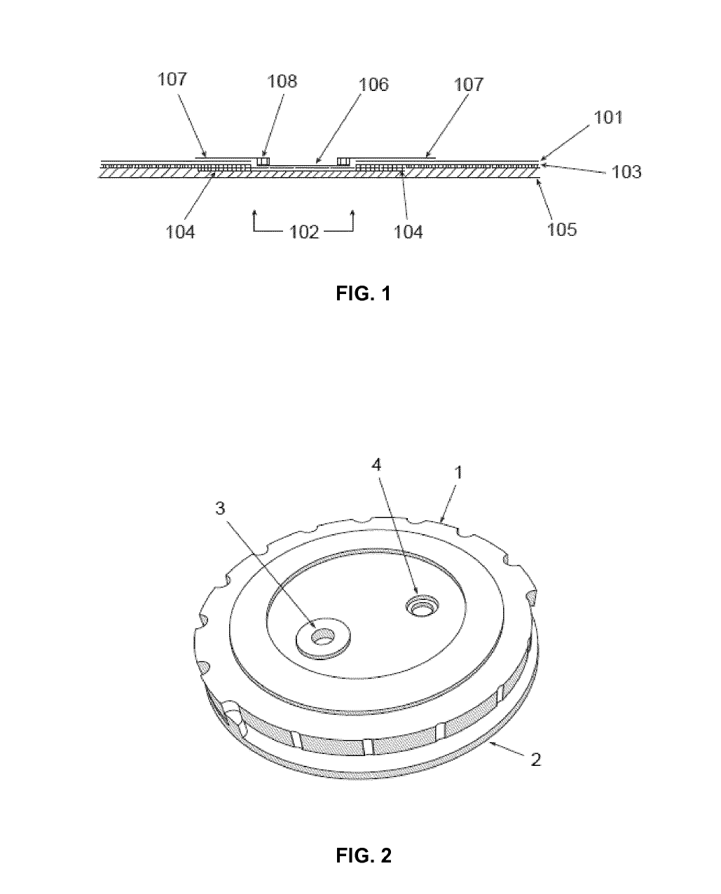

A non-limiting example of a simple device for performing a vertical flow assay of the present invention is shown in FIG. 1. FIG. 1 is a vertical section of such a device illustrating in particular the sequence of different layers of filter and adsorbent materials required for performing the assay.

In an upper square disc layer 101 a central circular aperture 102 is provided. Underneath the said square disc on its lower surface, a thin layer 103 of glue is provided in order to fix a circular piece of a filter 104 with a suitable pore size, to the lower side of said disc layer 101, with its center in the middle of the central aperture 102 of said disc. The glue layer 103 also fixes to the lower side of the said disc 101 a square absorbent pad 105 of about the same size as that of the disc 101. In the central hole 102 of the disc 101, on top of the underlying filter 104, a disc of a suitable net filter 106, attached to a ring 108 is inserted into the central aperture and is removably fastened to the upper side of disc 101 by means of and an adhesive tape 107 fixed to the upper side of the ring 108. In tape 107 a central aperture is formed which allows adding the sample to be analyzed, and washing reagents on top of the net filter 106. Filter 106 may be removed from the device after sample addition and washing is completed by pulling off the tape 107. Washing buffer and further reagents may then be added to the remaining “opened” device through aperture 102 directly onto filter 104. The test result (as for example a color reaction, may be visually inspected and further analyzed through said aperture 102.

FIGS. 2, 3 and 4 illustrate another non-limiting embodiment of a vertical flow assay device.

As can be taken from FIG. 2, the assay device comprises an upper casing element 1 and a lower casing element 2. The upper casing element 1 has a first opening 3, in the depicted case a sample feed opening, and a second opening 4, in the depicted case a reading opening 4. The upper 1 and the lower casing element 2 are assembled on top of each other. The assembly comprising the upper 1 and lower casing element 2 has the shape of a flat round disc, i.e. the radius of the resulting assembly is larger than the thickness of the disc.

In an optional variant of this embodiment, a card 10 is provided with a hole 10a, which is suited to take up the assembled assay device. Particularly, the shape of the hole 10a of the card 10 is formed in such a way that it is suited to interlock with at least one portion of the lower casing element 2. The hole 10a may also comprise a notch, which is suited to hold the lower casing element 2 in place and to prevent it from a rotation with respect to the card 10.

In another variant of this embodiment, an explanatory imprint may be provided on the card 10, e.g., instructions for the use of the assay device or information to facilitate the quantification of measurements using the assay device, as for example reference colored spots as explained above.

With reference to FIG. 4, a cross-section of the assay device according to FIG. 2 is described.

The upper 1 and lower casing element 2 comprise an upper 1a and a lower testing compartment inner surface 2a, which are facing each other and extend essentially in parallel to each other. The upper 1 and lower casing element 2 are furthermore formed in such a way that a testing compartment is formed between them. The upper 1a and lower testing compartment inner surface 2a form the top and bottom surfaces of a cylindrical testing compartment.

The testing compartment is provided with an upper membrane layer 6 and a lower absorbent layer 7, which are arranged on top of each other and extend essentially in parallel to the upper 1a and the lower testing compartment inner surface 2a. In this embodiment, the testing compartment is essentially filled out by the upper membrane layer 6 and the lower absorbent layer 7, i.e. said layers as inserted in form-locking manner. In further embodiments, the upper membrane layer 6 is spaced apart from the upper testing compartment inner surface 1a while still being inserted in the lower testing compartment in form-locking manner.

The second, lower testing chamber inner surface 2a is provided with a protrusion 2b that is suited to hold the lower absorbent layer 7 in place by restricting its mobility, in particular by inhibiting any mobility during the rotational movement of the assay device during the assay procedure, particularly by completely avoiding rotational movement inside the testing compartment. In other embodiments of the invention, the protrusion 2b further extends into the testing compartment and is suited to also hold the upper membrane layer 6 in place. In other embodiments, the lower absorbent layer and/or the upper membrane layer are kept in place alternatively or additionally by other attachment means, e.g., by glue.

The assembly further comprises a filter layer 5, which in the depicted embodiment is arranged inside a recession 5a of the upper testing compartment inner surface 1a right below the first opening 3. The filter layer 5 is attached to the upper casing element 1, particularly to restrict its motion with respect to the upper casing element 1. In the depicted case, the filter 5 is glued to the upper casing element 1 such that the first opening 3 is covered on the side facing the testing compartment.

In this embodiment, the second opening 4 of the upper casing element 1 serves primarily as a reading opening 4, wherein the second opening offers direct optical access from outside through the upper casing element 1 to the testing compartment and an unobstructed view of the upper membrane layer 6. The second opening is also used for the addition of reagent solutions and washing solutions on top of the membrane layer carrying the analyte (like particular blood cells) retained on the surface of said membrane layer 6.

In this embodiment, the lower absorbent layer 7 comprises an absorbent material for taking up lower molecular substances and liquid which are not retained by the upper membrane 6. The upper membrane layer 6 comprises a semi-permeable membrane retaining the analyte, in, particular blood cells suspected to carrying the analyte in said cells, or preferably, on the cell surface. Furthermore, the filter layer 5 comprises a semi-permeable membrane or preferably grid, permeable for non-agglutinated blood cells and smaller constituents of the sample, while retaining larger agglomerates of blood cell which have to be removed before the analytical detection reaction on the surface of the upper membrane is finally performed.

The assembly of the upper 1 and lower casing element 2 comprises an interlocking mechanism in which the upper casing element 1 takes up a portion of the lower casing element 2. Due to the round shape of the interlocking portions of the upper 1 and lower casing element 2, the upper 1 and lower casing element 2 may be rotated with respect to each other, wherein a rotational angle defines a position of the two casing elements 1, 2 to each other. Latches 12 are provided on the interlocking portion of the lower casing element 2, which are suited to hold the assembly of the upper 1 and lower casing element 2 firmly in place and leave essentially only a rotational degree of freedom for motion of the casing elements 1, 2 relative to each other.

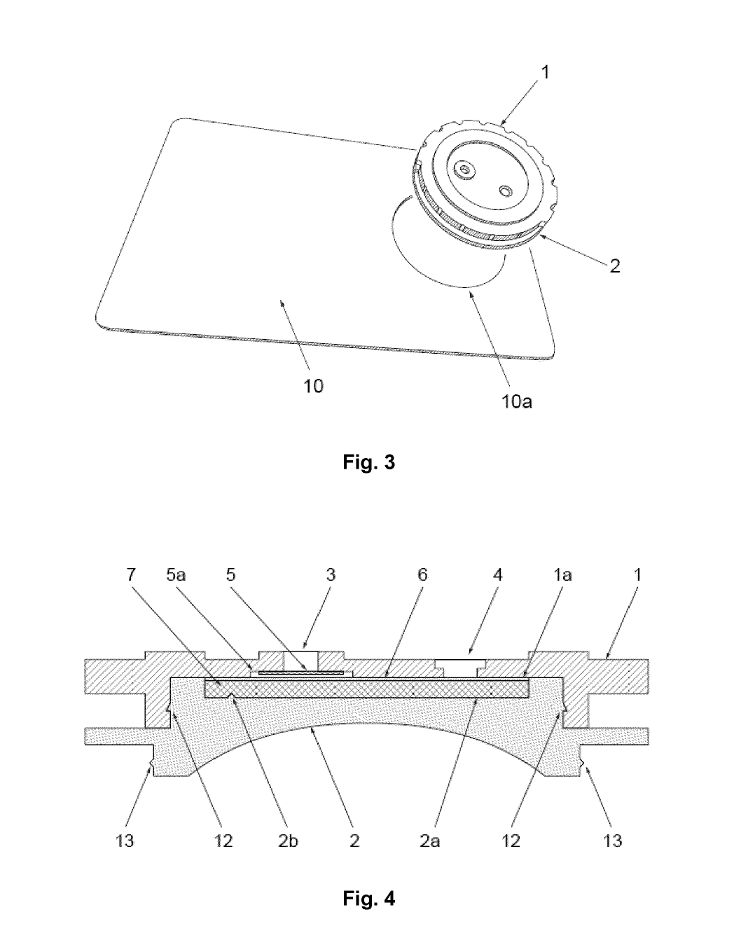

Furthermore, latches 13 are provided on a portion of the lower casing element 2 interlocking with the hole 10a in the card 10 as shown in FIG. 3.

The latches 12, 13 may be formed in different ways, as a person skilled in the art will appreciate. Furthermore, corresponding grooves are formed in the upper casing element 1 corresponding to the latches 12 of the lower casing element. Similar structures may be formed in the card 10 in order to facilitate the interlocking action with the lower casing element 2.

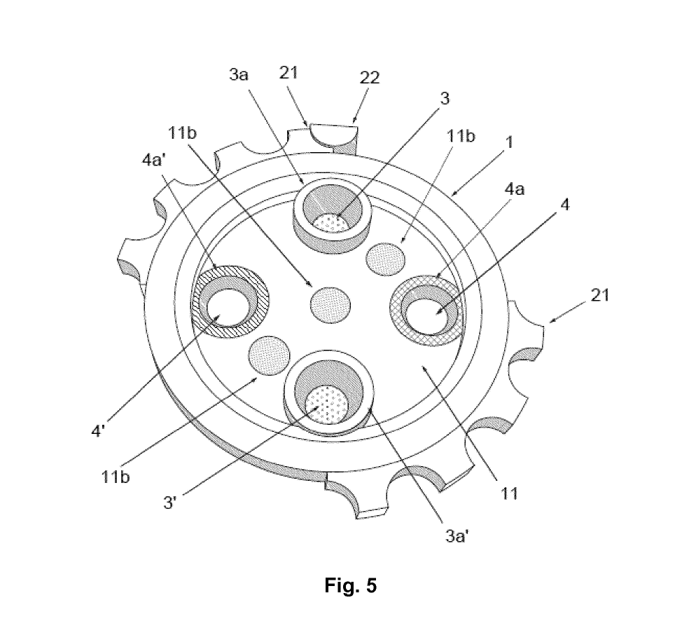

In FIG. 5 a top view of another non-limiting embodiment of an assay device is depicted. The configuration of the assay device is analogous to the structures described above with reference to FIGS. 2 to 4.

For simplification purposes, the lower casing element 2 is not depicted in FIG. 5 except for the rotation stop 22. The upper casing element 1 is provided with two rotation stops 21, which engage with the rotation stop 22 of the lower casing element 2 in the first and second configuration, respectively. Herein, the first configuration of the assay device is shown and the second configuration can be reached by rotating the upper casing element 1 anticlockwise with respect to the lower casing element 2 towards the second extreme rotation angle that is defined by the rotation stops 21, 22.

A first 3, 4 and a second pair 3′, 4′ of first 3, 3′ and second openings 4, 4′ are formed in the upper casing element 1, wherein the first openings 3, 3′ are provided with ridges 3a, 3a′ around their respective circumference. Also, the filter layers 5, 5′ that extend across the first openings 3, 3′ on the bottom side of the upper casing element 1 are shown by a hatching inside the first openings 3, 3′. The pairs of openings 3, 4, 3′, 4′ are arranged in such a manner that, in the second configuration (after rotation), the positions of the second openings 4, 4′ relative to the lower casing element, which is represented by its rotation stop 22, will be essentially the same as the positions of the first openings 3, 3′ in the first configuration.

Furthermore, the label 11 is arranged on the top surface of the upper casing element 1 and the explanation imprint 11b is visible to a user of the assay device. Also, circumferential imprints 4a, 4a′ are provided in the label 11 around the second openings 4, 4′. Different hatching of the circumferential imprints 4a, 4a′ illustrate the differences in coloring that, e.g., help the user to easily differentiate the individual second openings 4, 4′ from each other or give a reference color for the interpretation of a colorimetric read-out of the assay.

C.4.2 Performing the Assay Method of the Invention

C.4.2.1 CD4 Assessment

This embodiment refers to the assessment of CD4 receptors on CD4+ lymphocytes, in particular T helper cells.

In said embodiment of the invention, the labelled antibody is reactive to the CD4 receptor, and said label is constituted by an enzyme or colored or fluorescent particle. If an enzyme is used as label, a preferred embodiment is characterized by the subsequent exposure to the said filter of a substrate forming a colored substance, preferentially a precipitating colored substance, preferably a precipitating colored substance.

The correlation between color or fluorescence generated in the method of the present invention and the concentration of said class of receptor molecules, can be performed as follows: There is a direct relationship between the amount of the said specific receptor molecules and the color to be measured, since the amount of colored particles bound relates to the amount of said specific receptor molecules present in the sample to be tested. This color is then detectable either visually with comparison to preevaluated, pre-calibrated and/or predetermined coloristic diagrams or by measurement of the amount of color by electronic color detectors either freely available on the marked or the one developed for the present invention.

Measurement instruments used are easily calibrated and adjusted to colored substances or immunoparticles used, their color scheme and detection range needed. In calibration for detection instruments a known amount of analyte is used, giving a good ratio of background vs. signal, and will allow users to be provided with exact calculated readouts.

If an enzyme—including but not limited to peroxidase enzymes or alkaline phosphatase—is used in the place of colored or fluorescent substances, a color generating or a fluorescent generating substrate for said enzymes are used. Measurements of two components with different color deposited on a filter by measurement of reflectance at two and more wavelengths is well known to the skilled man of the art. It was already described in Clinical Chemistry 43:12 2390-2396 (1997) in the article “Glycohemoglobin filter assay for doctors' offices based on boronic acid affinity principle” by Frank Frantzen et al., in U.S. Pat. No. 5,702,952 by Erling Sundrehagen and Frank Frantzen, and in U.S. Pat. No. 5,506,144 by Sundrehagen and Frantzen. Frantzen et al used a specialized reflectometer measuring reflectance (% R) at 620 and 470 nm. Measurements at these wavelengths were used to quantitate the blue-colored boronic acid conjugate and red hemoglobin (Hb), respectively. The instrument automatically performed Kubelka-Munk transformations (Kubelka P. New contributions to the optics of intensely light scattering materials. J Opt Soc Am 1948; 38:448-57) to linearize the recorded reflectance data. A “Portable rapid diagnostic test reader” is described in EP 2 812 675 and a “Spectroscopic sensor on mobile phone” is described in US 2006/0279732. Today the camera function on the mobile phone is commonly used for reflectometric measurements of filter based test devices in diagnostic medicine.

Such systems are also described in EP 0 953 149 (B1) by Sundrehagen and Bremnes. Many companies today deliver reflectometric scanning instruments or digital camera imaging software for measuring intensity and wavelength of reflected light from test spots on diagnostic devices, comprising software for calibration for computing the concentration on samples from intensity and wavelength of reflected light. The Scansmart system from Skannex AS, Oslo, Norway, is an example of an automated and dedicated system for this application, which has been sold for reflectometry spot intensity measurement for vertical flow tests to customers in Norway. Also standard “smartphones” with digital cameras can be used to obtain digital images of the color signal obtained. Typically, the digital images are then uploaded into Adobe Photoshop electronic program. This method allows graphic presentation of the results. This method also allows the determination of when the signal is strongest versus when the background is lowest. Standardization and calibration of the signals can be obtained by using reference spots with known intensity and concentration of the analyte to be measured.

If enzymatic color system generation is used, then kinetic measurements can be employed, and the measurement can be performed using a “video” mode.

The software Adobe Photoshop Elements 13© and the program “Eyedropper tool” to determine HSL and Red, Green and Blue and other color schemes to determine color of uploaded images. The HSL (hue, saturation and lightness) scheme provides a device-independent way to describe color. Especially instructive is http://www.handprint.com/LS/CVS/color.html on the internet. (July 2015).

In a special embodiment of the present invention, reference colored spots are placed or fastened in close proximity to the membrane with immobilized antibodies or bother binding molecules or fragments thereof, preferentially on the holder of the assay membrane. As a part of the measurements of the assay of the present inventions, these reference spots are measured as well. The measurement of said reference spot can—by the software of the measurement instrument, be used to compensate for instrument-to-instrument and other hardware variations, to increase the overall accuracy of the assay.

These reference spots may define color scale for each color in the analytical measurement. The instrument, e.g. the camera on a mobile telephone take a picture or a series of pictures of the surface to be measured, and also the reference spots on the device. Different software programs can convert the pixels measured into numeric values and define colour rooms in different numeric system. Very common is the RGB (Red Green Blue) colour room. The RGB color model is an additive color model in which red, green, and blue light are added together in various ways to reproduce a broad array of colors. The name of the model comes from the initials of the three additive primary colors, red, green, and blue. (Wikipedia 16 Jul. 2016)

HSL and HSV are the two most common cylindrical-coordinate representations of points in an RGB color model. The two representations rearrange the geometry of RGB in an attempt to be more intuitive and perceptually relevant than the cartesian (cube) representation. Developed in the 1970s for computer graphics applications, HSL and HSV are used today in color pickers, in image editing software, and less commonly in image analysis and computer vision.

A very modern and free software package in use today to measure and analyze color spots and give them numerical values in a color room, is GIMP. GIMP /gimp/ (GNU Image Manipulation Program) is a free and open-source raster graphics editor used for image retouching and editing, free-form drawing, resizing, cropping, photo-montages, converting between different image formats, and more specialized tasks. See www.gimp.org, where all aspects are explained.

The result is reported in the number of CD4-lymphocytes and CD8-lymphocytes per volume unit and/or as a ratio between the two numbers.

The CD4 assessment may be performed with a simple device as depicted in FIG. 1, as follows:
1. A whole blood sample was mixed with dilution buffer adapted to hypotonic lysis of erythrocytes as contained in the sample while not lysing the leucocytes. The dilution buffer also contains anti CD14 antibody in a form suitable to agglomerate CD14 monocytes.
2. After a short incubation time an aliquot of said mixture was transferred to the aperture 102 of the device of FIG. 1, and is immediately sucked into the coarse (nylon mesh) filter 106 inserted into said aperture and retaining agglutinated CD14 cells while letting pass through CD4+ T helper cells.
3. Thereafter, a wash solution was transferred to the aperture 102 of the filtration device and was sucked into the nylon mesh filter 106 and the filter 104 positioned below 106.
4. Thereafter, the nylon mesh filter 106 was removed from the device
5. Thereafter, a solution of anti-human CD4 receptor antibodies with detectable marker (like enzyme, coloured particles) was transferred to the aperture 102 of the filtration device and was sucked into the filter 104. After the solution was sucked into the filter 104 the antibody-conjugate was allowed to bind to the cells retained on the filter 104.
6. Thereafter, washing solution was transferred to the hole 102 of the filtration device, and was sucked into the filter 104.
7. Thereafter, if an enzyme is used as marker, the corresponding substrate was transferred to the hole 102 of the filtration device and was sucked into the filter 104.
8. A defined time (as for example 5 minutes) thereafter, the color developed was measured, as for example reflectometrically using a SkanSmart CE reader with software delivered by Skannex AS, Norway.
9. The reading was compared to a calibration curve stored in the software generated by calibration samples with known content of CD4+ lymphocytes, analyzed in identical experiments, and the content of CD4+ lymphocytes was calculated.

The CD4 assessment may be performed with a more advanced device as depicted in FIGS. 2, 3 and 4 as follows:
1. A whole blood sample was mixed with dilution buffer adapted to hypotonic lysis of erythrocytes as contained in the sample while not lysing the leucocytes. The dilution buffer also contains anti CD14 antibody in a form suitable to agglomerate CD14 monocytes.
2. After a short incubation time an aliquot of said mixture was transferred to the aperture 3 of the device of FIG. 4, and is immediately sucked into the coarse (nylon mesh) filter 5 positioned underneath said aperture 3 and retaining agglutinated CD14 cells while letting pass through CD4+ T helper cells which will be retained on the surface of the next filter layer 6.
3. Thereafter, a wash solution was transferred to the aperture 3 of the filtration device and was sucked into the nylon mesh filter 5, and the filter 6.
4. Thereafter, the nylon mesh filter 5 was removed by performing twisting the upper casing element 1 of the device (angle of rotation about 180°) so that opening 4 is now exactly in the previous position of opening 3 relative to the filter 6, i.e. the section of the filter where said CD4+ helper cells are adsorbed on the filter.
5. Thereafter, a solution of anti-human CD4 receptor antibodies with detectable marker (like enzyme) was transferred to the aperture 4 of the filtration device and was sucked into the filter 6. After the solution was sucked into the filter 6 the antibody-conjugate was allowed to bind to the cells retained on the filter 6.
6. Thereafter, washing solution was transferred to the hole 4 of the filtration device, and was sucked into the filter 6.
7. Thereafter, if an enzyme is used as marker, the corresponding substrate was transferred to the hole 4 of the filtration device and was sucked into the filter 6.
8. A defined time (as for example 5 minutes) thereafter, the color developed was measured, as for example reflectometrically using a SkanSmart CE reader with software delivered by Skannex AS, Norway.
9. The reading was compared to a calibration curve stored in the software generated by calibration samples with known content of T-cell associated CD4 receptor molecules, analyzed in identical experiments, and the content of T-cell associated CD4 receptor molecules was calculated.

If the detectable marker is for example a colored particle, then step 7 and the color development according to step 8 is not of course not necessary.

The CD4 assessment as described above for the more advanced device as depicted in FIGS. 2, 3 and 4, may in analogy also be performed with a device depicted in FIG. 5 where two blood samples may be assessed simultaneously. The angle of rotation of the uppercasing element 1 is in this case about 90°.

C.4.2.2 CD4 and CD8 Assessment

The assessment may be performed in analogy to the assessment of CD4, as described above by applying a device of FIG. 1 or a device as depicted in FIGS. 2 to 4 comprised the following steps:

Steps 1 to 4 and 6 may be performed in identical manner.

In Step 5 a suspension anti-CD4 antibodies anti-CD8 antibodies conjugated each conjugated to different, distinguishable markers, as for example latex particles of different color (like to red carboxylated latex and blue carboxylated latex) was transferred to the device and sucked into the filter.

Immediately thereafter, the color on the filter 104 or 6 was measured reflectometrically using a standard Apple i-Phone telephone and its inbuilt flash-light. Simultaneously two for example red (a weak and a strong) and two for example blue color dots (a weak and a strong) (depending on the color of the latex particles, for example also placed on top of the device was depictured. For all five spots, the BGR file obtained (see above) was used (by converting the files to gray scale) the place and the limits of the dots were decided. By the GIMP program (see above), all the pixels were transformed to HSV color values. The maximum and the minimum responses with respect to the two blue color dots defined the blue color room, and the maximum and the minimum responses with respect to the two red color dots defined the red color room.

The HSV value from the test spot (with both red and blue articles) was then interpolated into the red and the blue HSV color rooms, and HSV values for all pixels were calculated and normalized.

The obtained normalized values were then compared to the values obtained with the calibrating samples of known CD4 and CD8 positive lymphocytes (who had also been analyzed with for example a conventional Becton Dickinson Excalibur Flow Cytometry system), which had been stored in the calibrating file of the computer in the i-Phone system, and the results were reported on the display and in the electronic output. The result is reported in numbers per volume unit CD4-lymphocytes, CD8-lymphocytes per volume unit and as a ratio between the two.

The CD4 and CD8 assessment as described above for the more advanced device as depicted in FIGS. 2, 3 and 4, may in analogy also be performed with a device depicted in FIG. 5 where two blood samples, one for CD4 and the other for CD8, may be assessed simultaneously in different pairs of openings (3,4 and 3′, 4′). The angle of rotation of the uppercasing element 1 is in this case about 90°.
1. A whole blood sample was mixed with dilution buffer adapted to hypotonic lysis of erythrocytes as contained in the sample while not lysing the leucocytes. The dilution buffer also contains anti CD14 antibody in a form suitable to agglomerate CD14 monocytes.
2. After a short incubation time an aliquot of said mixture was transferred to the aperture 3 and 3′ of the device of FIG. 5, and is immediately sucked into the coarse (nylon mesh) filter 5 positioned underneath said aperture 3, 3′ and retaining agglutinated CD14 cells while letting pass through CD4+ T helper cells which will be retained on the surface of the next filter layer 6. (as for the CD8 assessment CD14 cell would not disturb the assay the filter 5 might also be omitted for the CD8 sample opening).
3. Thereafter, a wash solution was transferred to the apertures 4, 4′ of the filtration device and was sucked into the nylon mesh filter 5, and the filter 6.
4. Thereafter, the nylon mesh filter 5 was removed by performing twisting the upper casing element 1 of the device (angle of rotation about 90°) so that openings 4, 4′ are now exactly in the previous position of openings 3, 3′ relative to the filter 6, i.e. the section of the filter where said CD4+ helper cells are adsorbed on the filter.
5. Thereafter, a solution of anti-human CD4 receptor antibodies with detectable marker (like immunoparticles of a first color or enzyme) was transferred to the aperture 4 of the filtration device and a solution of anti-human CD8 receptor antibodies with detectable marker (like immunoparticles of a second color or enzyme) was transferred to the aperture 4′ of the filtration device; and each liquid was sucked into the filter 6. After the solution was sucked into the filter 6 the antibody-conjugates were allowed to bind to the cells retained on the filter 6 at said two different spots.
6. Thereafter, washing solution was transferred to the holes 4 and 4′ of the filtration device, and were sucked into the filter 6.

The further steps (color development in the case of an enzyme as marker) and measurement of colored spots may be performed as described above.

The following non-limiting examples further illustrate the present invention. Based on said teaching a person of ordinary skill in the art will be able to provide, without the need of undue experimentation or inventive effort, further embodiments of the invention.

### EXPERIMENTAL PART

### A. Materials and Methods

Unless otherwise stated all reagents and chemical compounds as used herein are of analytical grade.

### B. Examples

### Example 1: Preparation of a Nitrocellulose Filters with a Mean Pore Size 3, 5 and 8 μm and Nylon Net Filter with a Mean Pore Size of 30 μm

Whatman nitrocellulose filter (catalogue no 7193-002 for 3 μm pore size, cat no. 7195-004 for 5 μm pore size, and 10400112 for 8 μm pore size. cat. No) and Millipore Nylon Net Filter (Prod. No. NY3002500), were soaked for 4 hours at room temperature in a 1% bovine serum albumin solution in water. This blocking procedure was performed to avoid unspecific binding of protein and cells to the filters when later used in the vertical filtration devices.

Preferably, the nylon net filter can be supported in the periphery by a ring of polystyrene or another stiffer material, since the nylon net filter is a fluffy material. The said stiffer material should be glued, e.g. by Clearsol Casco glue, or melted to the nylon net filter to hinder liquids from leaking in between the ring and the nylon net filter (see filter (106) and ring (108) in FIG. 1 as described in Example 7, below).

### Example 2: Preparation of Polyclonal Antibodies Anti-Human CD14 Receptor Antibodies from Chicken Eggs

Human CD14 receptor, tailor-made from Novoprotein Inc, US, with the following amino acid sequence (SEQ ID NO:1)

| | |
|---|---|
| MNHKV HMELDDEDFR CVCNFSEPQP DWSEAFQCVS AVEVEIHAGG LNLEPFLKRV | 61 |
| DADADPRQYA DTVKALRVRR LTVGAAQVPA QLLVGALRVL AYSRLKELTL EDLKITGTMP | 121 |
| PLPLEATGLA LSSLRLRNVS WATGRSWLAE LQQWLKPGLK VLSIAQAHSP AFSCEQVRAF | 181 |
| PALTSLDLSD NPGLGERGLM AALCPHKFPA IQNLALRNTG METPTGVCAA LAAAGVQPHS | 241 |
| LDLSHNSLRA TVNPSAPRCM WSSALNSLNL SFAGLEQVPK GLPAKLRVLD LSCNRLNRAP | 301 |
| QPDELPEVDN LTLDGNPFLV PG | |

was suspended in Freund's complete adjuvants (FCA

Polyclonal anti human CD14 antibodies can be prepared by methods well known in the art, such as those described for example by Chase, M. W., 1967, in “Methods of Immunology and Immunochemistry”, ed. Williams, A. et al., M. W., pp. 197-209, Academic Press, New York. Briefly, animals of a suitable species (for example rabbits, goats, or sheep, or, preferably avian species, in particular poultry, like hens) are repetitively immunized with purified antigen in an appropriate adjuvant, for example Freund's complete or incomplete adjuvant. After immunization the animals are bled and the polyclonal antibodies are purified by methods such as for example ammonium sulfate or ammonium chloride precipitation, anionic exchange chromatography, immunoaffinity chromatography, and/or affinity chromatography.

Polyclonal avian antibodies routinely are obtained from egg yolk (and are therefore designated IgYs). Egg yolk, however, contains large amounts of lipids making their further use problematic. IgY can be isolated from egg yolk by using stepwise ammonium sulphate (for example 25 to 40%) and polyethylene glycol (PEG) precipitation. For initial purification also commercial IgY purification kits obtainable from Gallus Immunotch Inc, Cary, USA, or the Eggcellent Chicken IgY Purification Kit, obtainable from Pierce, Rockford, USA may also be employed considering the manufacturer's instructions.

Furthermore, the avidity of polyclonal antibodies may be further increased by using antibodies that ware purified by the use of antigen affinity purification methods, for example according to the teaching in “Affinity Purification of Proteins” downloaded from www.piercenet.com (April 2006) and incorporated by reference Affinity purification is described in more detail below.

Two to four hens were used for each immunization experiment. 0.1 mg peptide dissolved in 1 ml water was emulsified with equal volume of Freund's complete adjuvant and injected into the breast muscle of hens. The injection was repeated every 4 weeks. 10 weeks after the start of the injections, eggs were collected. The egg yolk was isolated from the eggs, and the IgY fraction from the egg yolk was isolated by ammonium chloride precipitation, in a conventional manner according to prior art methods of egg antibody isolation (see for example Larsson A, Baaloew R-M, Lindahl T, and Forsberg P-O in Poultry Science 72:1807-1812, 1993).

Further immunizations were performed every four weeks. After 10 weeks, eggs were collected and the egg yolk was isolated manually from the egg white. The total antibody fraction (the IgY fraction) from the egg yolk was isolated by ammonium chloride precipitation, in a conventional manner according to prior art methods of egg antibody isolation (see for example Larsson A, Baaloew R-M, Lindahl T, and Forsberg P-O in Poultry Science 72:1807-1812, 1993).

10 mg of highly pure human CD14 receptor was then immobilized on a HITRAP NHS-Active HP column from Amersham Pharmacia Biotech, following the prescription in the package insert of the column. The IgY fraction isolated from egg yolk was diluted to 2 mg/ml in phosphate buffered saline. 200 ml of this IgY solution was passed through the column, followed by 50 ml phosphate buffered saline with no IgY. The antibodies with specific affinity for the immobilized CD14 receptor was eluted with 35 ml of 0.1 M citrate buffer pH=3.0. The eluted specific anti-CD14 antibodies were dialyzed against phosphate buffered saline and concentrated to 3 mg/ml using an Amicon Centricon centrifugation filtration device with molecular weight cut-off of 30.000 Dalton.

### Example 3a: Conjugation of Anti-Human CD14 Antibodies to Carboxylated Polystyrene Particles

1 μm carboxylated polystyrene particles (product no. PC04N/10356) was purchased from Bangs Particles, USA. 31 mg of chicken anti-human CD14 antibodies, prepared according to Example 2 above, were dialyzed to 20 ml buffer (pH=9.5, 5 mM borate, 7.5 mM sodium chloride). 300 mg of said carboxylated polystyrene particles were washed by centrifugation and suspended in 20 ml water. 12.5 mg EDC (1-Ethyl-3-(3-dimethylaminopropyl)carbodiimide) (Sigma, US) was dissolved in the particle suspension. The antibody solution was added to the latex suspension, and stirred for 5 hours. The particles in the suspension were then washed 4 times with 1 mM NaCl, 0.5 mM sodium borate, 0.025% Tween 20, 0.5 mM glycine, pH=9.5. This stock solution was then diluted 1:3 in 30 mM borate buffer (pH 9.1-9.3 with 150 mM sodium chloride, 0.1% Tween 20, 0.5 mg/ml PSA (porcine serum albumin) and 0.1% ProClin™ 950 biocide).

### Example 3b: Polymerization of Anti-CD14 Antibodies

10 mg of anti CD14 IgY antibodies, prepared as described in Example 2 above, was added dropwise 1 ml of a PBS solution of 4 mg of dithiobis (sulfosuccinimidyl propionate) (manufactured by Pierce Corp., hereinafter referred to as DTSSP) with stirring at room temperature. After stirring the mixed solution at 35° C. for 30 minutes, the mixed solution was filtered through a Sepharose gel (manufactured by Pharmacia Fine Chemical Inc., Sephadex G25M column). This gave approximately 6 ml of the PBS solution containing IgY polymer (hereinafter referred to as IgYagg.) This procedure was in analogy to the polymerization procedure described in EP-A-0 957 363.

### Example 3c: Monoclonal Anti-CD14 Antibodies Coupled to Sepharose Particles

10 mg of monoclonal anti-human CD14 antibody (origin mouse) (product no. 3110 from Diatec Inc., Oslo, Norway), was dialyzed against 0.2 M sodium hydrogen carbonate, 0.5 M sodium chloride pH 7.9. 37.5 mg of egg albumin from Norwegian Antibodies Inc. in Norway was dialyzed against 0.2 M sodium hydrogen carbonate, 0.5 M sodium chloride, pH 7.9.

6 ml of NHS-activated Sepharose (product no. 17-0906-01 from GE Health Care Life Sciences) was washed with 1 mM HCl, allowed to settle, and then washed three times with purified water.

12.5 mg of dialyzed egg albumin and 10 mg of the dialyzed anti-human CD14 was mixed, and then mixed with the 6 ml of NHS-activated Sepharose. The suspension was agitated 4 hours at room temperature. Then the remaining 25 mg of dialyzed albumin was added, and the suspension was agitated another 4 hours at room temperature. The Sepharose is allowed to settle, and then washed 3 times with 0.1 M Tris-HCl, pH 8.5, 0.15 M NaCl. In the end, the volume of the coupled Sepharose was 20 ml.

### Example 4a: Sample Dilution Buffer Containing Anti-CD14 Antibodies

10 mM NaCl with 0.5 mg/mL BSA and 0.1% proclin, pH 7.5 And this is the buffer we have used for hypotonic lysis of erythrocytes without lysis of leucocytes.

Anti-CD14 antibodies made according to Example 2, above, was added to said buffer solution at an amount sufficient to bind and aggregate all monocytes in a whole blood volume which shall be analyzed for CD4 receptors using the method of the present invention. If, for example, 20 μl whole blood will be analyzed, and a volume of 400 μl of sample dilution buffer shall be used, an amount of anti-CD14 antibodies which will bind all monocytes of 20 μl blood must be present in 400 μl of the sample dilution buffer. A titration of the amount necessary can be done by setting up a dilution series of said antibodies in 400 μl samples of dilution buffer, and test by applying the following method.

The method to be used in order to find out whether the amount of anti-CD14 present in or to be added to the hemolyzing solution is sufficient to bind all CD14 molecules in the mixture comprises the following steps:
1. Make a lysis buffer by mixing 20 μl whole blood with a known high amount of monocytes and CD14 receptors with 400 μl of a solution of 10 mM NaCl, 0.5 mg/ml bovine serum albumin having a pH=7.5 and comprising anti-CD14 antibodies made according to Example 2. The amount of anti-CD14 antibodies comprised was varied according to the description below.
2. Agitate the mixture for 120 minutes.
3. Filter the mixture through a Millipore Nylon Net Filter Prod. No. NY3002500 according to Example 1.
4. Test presence of CD14 in the filtered solution by
   a) placing a nitrocellulose filter disc diameter of 5 mm with a pore size of 3 μM and blocked according to Example 1 above, on top of an absorbance pad (Absorbent CF7 from Whatman—article no. 8117 in a filter holder.
   b) placing 50 μl of the filtered sample (see above) on the nitrocellulose filter, and sucking into the filter and the absorbent beneath, while white blood cells including monocytes left in the filtered sample will be retained on the nitrocellulose filter.
   c) washing the cells/nitrocellulose filter by applying 50 μl of a solution of 0.01 M Tris, 0.14 M NaCl 1 mg/ml bovine serum albumin, 0.1% Tween 20 with pH=7.4.
   d) applying an anti-human CD14 alkaline phosphatase conjugate (for this purpose an anti-human CD14 (same as in Example 3c) Product 3110 from Diatec AS, Oslo, had been conjugated to alkaline phosphatase (Diatec prod. No. 3119, clone 18D11 anti-human CD14 antibody conjugated to); the conjugated was diluted 1:100 in Kementech AP Stabil solution (catalogue no. 4-40-H) with 0.1% Tween; 60 μl of this mixture were applied on the said nitrocellulose filter) and incubating for 3 minutes.
   e) washing the cells/nitrocellulose filter by applying 2×60 μl of a solution of 0.01 M Tris, 0.14 M NaCl, 1 mg/ml bovine serum albumin, 0.1% Tween 20 with pH=7.4.
   f) applying 20 μl of Seramun Purple S-800-NBT on the remaining cells and the nitrocellulose filter <<and let it drain into the absorbent.
   g) developing color for 10 minutes.

If no color developed, then there had been enough of CD14 binding molecules in the lysis buffer described above. If significant color developed, more CD14-binding substances had to be added to the lysis buffer described above.

### Example 4b: A Sample Dilution Buffer with Polymerized Anti-CD14 Antibodies

A solution of 10 mM NaCl with 0.5 mg/mL BSA and 0.1% proclin, pH 7.5, was made. Polymerized anti-CD14 antibodies made according to Example 3b, above, was added to said buffer solution at an amount sufficient to bind and aggregate all monocytes in a whole blood volume which shall be analyzed for CD4 receptors using the method of the present invention. If, for example, 20 μl whole blood will be analyzed, and a volume of 400 μl of sample dilution buffer shall be used, an amount of polymerized anti-CD14 antibodies which will bind all monocytes of 20 μl blood must be present in 400 μl of the sample dilution buffer.

The method to be used in order to find out whether the amount of anti-CD14 present in or to be added to the hemolyzing solution is sufficient to bind all CD14 molecules in the mixture comprises the following steps:
1. Make a lysis buffer by mixing 20 μl whole blood with a known high amount of monocytes and CD14 receptors with 400 μl of a solution of 10 mM NaCl, 0.5 mg/ml bovine serum albumin having a pH=7.5 and comprising polymerized anti-CD14 antibodies made according to Example 3b above. The amount of polymerized anti-CD14 antibodies comprised was varied according to the description below.

Steps 2 to 4 are performed as described for Example 4a.

If no color developed, then there had been enough of CD14 binding molecules in the lysis buffer described above. If significant color developed, more CD14-binding substances had to be added to the lysis buffer described above.

### Example 4c: A Sample Dilution Buffer with Anti-CD14 Antibodies Conjugated to Sepharose Particles

A dilution buffer solution of 10 mM NaCl with 0.5 mg/ml BSA and 0.1% proclin adjusted to 7.4, was made. Anti-CD14 antibodies immobilized on Sepharose particles made according to Example 3c above, were added to said buffer solution at an amount sufficient to bind all or substantially all monocytes in a whole blood volume which shall be analyzed for CD4 receptors using the method of the present invention. If, for example, 20 μl whole blood will be analyzed, and a volume of 400 μl of sample dilution buffer shall be used, an amount of anti-CD14 antibodies conjugated to Sepharose particles according to Example 3 c above, and which will bind all monocytes of 20 μl blood must be present in 400 μl of the sample dilution buffer.

The method to be used in order to find out whether the amount of anti-CD14 present in or to be added to the hemolyzing solution is sufficient to bind all CD14 molecules in the mixture comprises the following steps:
1. Make a lysis buffer by mixing 20 μl whole blood with a known high amount of monocytes and CD14 receptors with 400 μl of a solution of 10 mM NaCl, 0.5 mg/ml bovine serum albumin having a pH=7.5. and comprising monoclonal anti-CD14 antibodies coupled to agarose particles, made according to Example 3c above. The amount of anti-CD14 agarose particles was varied according to the description below.

Steps 2 to 4 are performed as described for Example 4a.

If no color developed, then there had been enough of CD14 binding molecules in the lysis buffer described above. If significant color developed, more CD14-binding substances had to be added to the lysis buffer described above.

### Example 5: Washing Buffer

A solution of 0.14 M sodium chloride, 1 g/l of Tween 20 (Sigma), 0.01 M of 2-amino-2-hydroxymethyl-propane-1,3-diol (Sigma), 1 gram bovine serum albumin (Sigma) per liter and 1 g/l of Proclin 300, pH adjusted to 7.4 was prepared.

### Example 6: Solution of Enzyme-Conjugated Monoclonal Mouse Anti-Human CD4 Receptor Antibodies

Alkaline phosphate enzyme conjugated to EDU-2 clone of monoclonal anti-human CD4 receptor was purchased from Diatec AS, Oslo, Norway. It was supplied in 0.5 mg/ml. In the working solution it was diluted 1:100 in a Kementech AP Stabil solution (catalogue number 4-40 H) and Tween was added to 0.1% vol/vol in the final solution.

### Example 7: A Vertical Flow Assay Device According to FIG. 1

A vertical filtration device was formed around a 0.20 mm thick square polystyrene disc measuring 22×22 mm (101).

In the center of the polystyrene disc a 5 mm circular hole (102) is punched out with a standard punching instrument.

Underneath the said square polystyrene disc, a thin layer (103) of Clearseal Casco glue was smeared using a small brush. A circular piece of a nitrocellulose (104) with a mean pore size of 3, 5 or 8 μm, prepared according to Example 1 above, having a diameter of 10 mm, is placed on the glue of said polystyrene disc, with its center in the middle of the central aperture (102) of said polystyrene disc.

Thereafter, the whole glue side of the said polystyrene disc was covered by a 22×22 mm CF7 absorbent pad (105) (100% cotton linter material) from GE Health Care Life Sciences, and the glue was allowed to dry.

In the hole of the polystyrene disc (102), on top of the underlying nitrocellulose filter, a 5 mm diameter disc of a nylon net filter (106), according to Example 1, was fastened to the polystyrene disc by the use of Clearseal Casco glue and a ring of polystyrene (108) and a 22×10 mm piece of adhesive tape (107), with a central aperture of 3 mm in the adhesive tape (107).

### Example 8: A Vertical Flow CD4 Assay Using Enzyme Immunoconjugates

The assay performed with a device of FIG. 1 comprised the following steps:
1. A whole blood sample of 25 μl was mixed with 500 μl dilution buffer according to Example 4c above.
2. One minute thereafter, 50 μl of said mixture was transferred to the aperture of the adhesive tape (107) of the filtration device according to Example 7 above, and is immediately sucked into the nylon mesh filter (106).
3. Thereafter, 50 μl of wash solution according to Example 5 above was transferred to the aperture of the adhesive tape (107) of the filtration device according to Example 7 above, and was sucked into the nylon mesh filter.
4. Thereafter, the adhesive tape (107) and the nylon mesh filter (106) was removed by ripping off the said adhesive tape.
5. Thereafter, 50 μl of solution of enzyme conjugated monoclonal mouse antihuman CD4 receptor antibodies according to Example 6 above, was transferred to the hole of the polystyrene disc (102) of the filtration device according to example 7 above, and was sucked into the nitrocellulose filter (104). After the solution was sucked into the filter the antibody-conjugate was allowed to bind to the cells for 2 minutes.
6. Thereafter, 100 μl of washing solution according to Example 5 above was transferred to the hole of the polystyrene disc (102) of the filtration device according to Example 7 above, and was sucked into the nitrocellulose filter (104).
7. Thereafter, 20 μl of Seramun Purple S-008-NBT liquid enzyme substrate Seramun GmbH was transferred to the hole of the polystyrene disc (102) of the filtration device according to Example 7 above, and was sucked into the nitrocellulose filter (104) and color developed for 5 minutes.
8. Five minutes thereafter, the color developed was measured reflectometrically using a SkanSmart CE reader with software delivered by Skannex AS, Norway.
9. The reading was compared to a calibration curve stored in the software generated by calibration samples with known content of T-cell associated CD4 receptor molecules, analyzed in identical experiments, and the content of T-cell associated CD4 receptor molecules was calculated.

Alternative Procedure:

In order to avoid the use of time-consuming enzymatic signal generation, the said antihuman CD4 antibody can be coupled to a colored substance or a fluorescent substance which can be read immediately after step 6, above. Colored immunoparticles comprise antibodies or immunoreactive fragments thereof and particulate materials exhibiting a color. The particulate material can be coupled to the said antibodies or fragments by physical absorption or covalent coupling, often with a spacer or bridging molecules. The colored material may be constituted—but is not limited—to pigments in or on latex particles or polymer particles, which can be made from many different materials, or metal colloids like gold colloids or ferric colloids or carbon particles. Such colored particles are described in the prior art and are well known by the skilled man of the art. They are typically purchased from suppliers like Merck France, Life technologies (US) and/or Bangs Laboratories (US). Polymer particles are supplied in all sizes and colors, also as fluorescent particles. The size and color intensity of the particles must be adjusted to the sensitivity and the capacity needed for the assay methods, as well as to the pore size of the membranes used in the product of the invention.

As mentioned above, the present invention employs a membrane for capturing a specific group of cells, and the measurement of receptors associated with said groups of cells. Depending on the size of the cells or the specific group of cells, the said membrane has a pore size adapted to capture said group of cells, and allowing other smaller particles to pass through the said membrane. In one embodiment of the present invention, the specific group of cells are T lymphocytes, and a suitable pore size of said membrane for capturing the T-cells are membranes with an average pore size 1-10 μm, preferentially 3-9 μm, and even more preferred 3-5 μm, allowing smaller particulate materials contained in the sample material after the hypotonic to pass through the membrane. In preferred embodiments of the present invention, the present invention employs colored immunoparticles in a size generating a good signal from the colored immunoparticles, but small enough to pass through the said membrane which captures the said specific group of cells. Typically, the present invention employs immunoparticles sized from 60 to 400 nm, more preferred from 80 to 300 nm, even more preferred 95 to 200 nm in diameter.

### Example 9: Anti-CD4 Antibodies Conjugated to Blue Carboxylated Latex

In one embodiment of the present invention, blue carboxylated latex particles from Millipore, Europe (Prod. No. PSI 90-91), with a mean diameter of 117 nm, were employed. 5 mg EDU-2 clone of monoclonal anti-human CD4 receptor antibodies, from Diatec AS, Norway, were dialyzed to 5 ml buffer (pH=9.5 in 5 mM borate, 7.5 mM sodium chloride). 23.4 mg of said carboxylated blue latex particles were washed by centrifugation and are suspended in 2 ml water. 0.8 mg EDC (Sigma, US) was dissolved into the particle suspension and the antibody solution was mixed with the latex suspension, and stirred for 5 hours. The particles in the suspension were then washed 4 times with 1 mM NaCl, 0.5 mM sodium borate, 0.025% Tween 20, 0.5 mM glycine, pH=9.5. This stock solution was then diluted 1:3 in 30 mM borate buffer (pH 9.1-9.3 with 150 mM sodium chloride, 0.1% Tween 20, 0.5 mg/ml PSA and 0.1% ProClin 950).

The signal strength of this preparation will vary somewhat from batch to batch, and the appropriate working solution was found by identifying the appropriate dilution of the stock solution in a solution of 15 mM TRIS, 10 mM borate, 15 mM NaCl with 0.1% Tween and 1 mg/ml bovine serum albumin, pH adjusted to 7.4.

### Example 10: A Vertical Flow CD4 Assay Using Blue Latex Immunoparticles

The assay performed with a device of FIG. 1 comprised the following steps:
1. A whole blood sample of 25 μl was mixed with 500 μl dilution buffer according to Example 4c above.
2. One minute thereafter, 150 μl of said mixture was transferred to the aperture of the adhesive tape (107) of the filtration device according to Example 7 above, and is immediately sucked into the nylon mesh filter (106).
3. Thereafter, 50 μl of washing solution according to Example 5 above was transferred to the aperture of the adhesive tape (104) of the filtration device according to Example 7 above, and was sucked into the nylon mesh filter.
4. Thereafter, the adhesive tape (107) and the nylon mesh filter (106) was removed by ripping off the said adhesive tape.
5. Thereafter, 50 μl of a suspension of anti-CD4 antibodies conjugated to blue carboxylated latex according to Example 9 above, was transferred to the hole of the polystyrene disc (102) of the filtration device according to Example 7 above, and was sucked into the nitrocellulose filter (104).
6. Thereafter, wait a predefined time before starting the washing step to let the antibody conjugated beads bind sufficiently to the cells.
7. Thereafter, 50 μl of washing solution according to Example 5 above was transferred to the hole of the polystyrene disc (102) of the filtration device according to Example 7 above, and was sucked into the nitrocellulose filter (104).
8. Immediately thereafter, the color on the nitrocellulose filter was measured reflectometrically using a SkanSmart CE reader with software delivered by Skannex AS, Norway.
9. The reading was compared to a calibration curve stored in the software generated by calibration samples with known content of T-cell associated CD4 receptor molecules, analyzed in identical experiments, and the content of T-cell associated CD4 receptor molecules was calculated.

Examples 8 and 10 employ the use of a sample dilution buffer according to Example 4c above, comprising anti-CD14 antibodies conjugated to polymer particles. Said sample dilution buffer can be replaced by a buffer containing non-conjugated anti-CD14 antibodies according to Example 4a, or polymerized antibodies according to Example 4b, or other well-known methods to make substances carrying many antibody molecules. In a non-limiting way, conjugating the antibodies to protein carrier molecules or to soluble polymer molecules, is another preferred option.

### Example 11: Anti-CD8 Antibodies Conjugated to Red Carboxylated Latex

Red carboxylated latex particles from Merck Estapore (Prod. No. 784 K1-010) with a mean diameter of 190 nm, were employed. 5 mg of UCHT-4 clone monoclonal antihuman CD8 receptor antibodies, from Diatec AS, Norway, were dialyzed to 5 ml buffer (pH=9.5, 5 mM borate, 7.5 mM sodium chloride). 35 mg of said carboxylated blue latex particles were washed by centrifugation and suspended in 2 ml water. 0.8 mg EDC (Sigma, US) was dissolved into the particle suspension and the antibody solution was mixed with the latex suspension, and stirred for 5 hours. The particles in the suspension were then washed 4 times with 1 mM NaCl, 0.5 mM sodium borate, 0.025% Tween 20, 0.5 mM glycine, pH=9.5. This stock solution was then diluted 1:3 in 30 mM borate buffer pH 9.1-9.3 with 150 mM sodium chloride, 0.1% Tween 20, 0.5 mg/ml BSA and 0.1% ProClin 950.

The signal strength of this preparation varied somewhat from batch to batch, and the appropriate working solution was found by identifying the appropriate dilution of the stock solution in 15 mM TRIS, 10 mM borate, 15 mM NaCl solution with 0.1% Tween and 1 mg/ml bovine serum albumin, pH adjusted to 7.4.

### Example 12: A Vertical Flow CD4 and CD8 Assay Using Blue and Red Latex Immunoparticles

The assay performed with a device of FIG. 1 comprised the following steps:
4. A whole blood sample of 20 μl was mixed with 400 μl dilution buffer according to Example 4c above.
5. One minute thereafter, 150 μl of said mixture was transferred to the aperture of the adhesive tape (107) of the filtration device according to Example 7 above, and is immediately sucked into the nylon mesh filter (106).
6. Thereafter, 50 μl of washing solution according to Example 5 above was transferred to the aperture of the adhesive tape (104) of the filtration device according to Example 7 above, and was sucked into the nylon mesh filter.
7. Thereafter, the adhesive tape (107) and the nylon mesh filter (106) were removed by ripping off the said adhesive tape.
8. Thereafter, 50 μl of a suspension of 50% anti-CD4 antibodies conjugated to blue carboxylated latex according to Example 9 above, and 50% anti-CD8 antibodies conjugated to red carboxylated latex according to Example 11 above, was transferred to the hole of the polystyrene disc (102) of the filtration device according to Example 7 above, and was sucked into the nitrocellulose filter (106).
9. After 3 minutes, 50 μl of wash solution according to Example 5 above was transferred to the hole of the polystyrene disc (102) of the filtration device according to Example 7 above, and was sucked into the nitrocellulose filter (106).
10. Immediately thereafter, the color on the nitrocellulose filter was measured reflectometrically using a standard Apple i-Phone telephone and its inbuilt flash-light. Simultaneously two red (a weak and a strong) and two blue color dots (a weak and a strong) placed on the device was depictured. For all five spots, the BGR file obtained (see above) was used (by converting the files to gray scale) the place and the limits of the dots were decided. By the GIMP program (see above), all the pixels were transformed to HSV color values. The maximum and the minimum responses with respect to the two blue color dots defined the blue color room, and the maximum and the minimum responses with respect to the two red color dots defined the red color room.
11. The HSV value from the test spot (with both red and blue articles) was then interpolated into the red and the blue HSV color rooms, and HSV values for all pixels were calculated and normalized.
12. The obtained normalized values were then compared to the values obtained with the calibrating samples of known CD4 and CD8 positive lymphocytes (who had also been analyzed with the Becton Dickinson Excalibur Flow Cytometry system), which had been stored in the calibrating file of the computer in the i-Phone system, and the results were reported on the display and in the electronic output. The result is reported in numbers of CD4-lymphocytes per volume unit, number of CD8-lymphocytes per volume unit and as a ratio between the two.

The disclosure of prior art documents as cited herein is incorporated by reference.

1. An assay method for assessing in a liquid whole blood sample or a sample derived therefrom one or more subclasses of blood cells of interest (BCol), each of which carrying a first cell surface marker (M1) for said sub-class of blood cells of interest, wherein said sample may additionally comprise disturbing blood cells (DBC), which carry at least one of said first cell surface markers (M1) as non-specific marker, and/or at least one free non-cell surface bound form of any of said first cell surface markers (M1), which method comprises:
(1) removing from said sample any disturbing blood cells (DBC);
(2) removing from said sample as obtained in step (1) any free, non-cell surface bound form of each of said first cell surface markers (M1); and
(3) assessing in the sample as obtained in step (2) each of said sub-classes of BCol, carrying said first cell surface marker (M1).

2. The method of claim 1, which is a vertical flow assay method.

3. The method of claim 1, wherein in step (1), said DBCs are removed by filtration.

4. The method of claim 3, wherein said DBCs are aggregated, which aggregates are retained by the filter applied in step (1).

5. The method of claim 4, wherein said DBCs are aggregated by means of immunoglobulin molecules which do not bind said BCol.

6. The method of claim 5, wherein said DBCs are aggregated by means of immunoglobulin molecules which bind to a second cell surface marker (M2) which is not present on the surface of said BCol.

7. The method of claim 5, wherein said DBC binding immunoglobulins are selected from free antibodies, polymeric antibodies or antibodies bound to the surface of solid particles, in particular polymer particles.

8. The method of claim 1, wherein in step (2) said non-cell surface bound form of said first cell surface marker (M1) is removed by filtration by applying a filter which is permeable for said non-cell surface bound form of said first cell surface marker (M1) but which retains said BCol.

9. The method of claim 1, wherein said assessment of step (3) is performed by means of immunoglobulin molecules reactive with said first cell surface marker (M1).

10. The method of claim 9, wherein said immunoglobulin molecules are labelled.

11. The method of claim 10, wherein said label is selected from an enzyme, a fluorescent or colored molecular marker or a fluorescent or colored particle.

12. The method of claim 1, wherein said BCol are selected from a sub-class of lymphocytes, in particular T-lymphocytes, and said DBCs are monocytes.

13. The method of claim 1, wherein said first cell surface marker (M1) is a T-lymphocyte marker (M1a), in particular the CD4 cell surface receptor molecule.

14. The method of claim 1, wherein said one or more sub-classes of blood cells of interest (BCol) to be assessed comprises CD4⁺ cells.

15. The method of claim 1, where said first cell surface marker (M1a) is CD4 and said first sub-class of cells is T-helper cells.

16. The method of claim 1, further comprising: (4) a second sub-class of BCol carrying a cell surface marker (M1b) different from said first cell surface marker (M1a).

17. The method of claim 16, wherein said cell surface marker (M1b) is a T-lymphocyte marker different from M1a, in particular the surface marker CD8 and said second sub-class of BCol comprises CD8⁺ cells.

18. The method of claim 17, where said surface marker (M1b) is CD8 and said second sub-class of cells is cytotoxic T-cells.

19. The method of claim 16, wherein the assessment of said second sub-class of BCol carrying said second cell surface marker (M1b) is performed in step (3) together with the assessment of said first subclass of BCol, carrying a first cell surface marker (M1a), in particular in the same sample.

20. The method of claim 16, wherein the assessment of said second sub-class of BCol carrying said marker (M1b) is performed separately.

21. The method of claim 20, further comprising:
optionally removing from said sample any disturbing macromolecular impurities which might disturb the assessment;
removing from said sample (optionally as obtained in step (4)) any free, non-cell surface bound form of said second cell surface markers (M1b); and
assessing in the sample as obtained in step (5) said sub-class of BCol carrying said cell surface marker (M1b).

22. The method of claim 21, wherein in step (5) said non-cell surface bound form of said first cell surface marker (M1b) is removed by filtration by applying a filter which is permeable for said non-cell surface bound form of said cell surface marker (M1b) but which retains said sub-class of BCol carrying (M1b).

23. The method of claim 22, wherein said assessment of step (6) is performed by means of immunoglobulin molecules reactive with said cell surface marker (M1b).

24. The method of claim 23, wherein said immunoglobulin molecules are labelled.

25. The method of claim 24, wherein said label is selected from an enzyme, a fluorescent or colored molecular marker or a fluorescent or colored particle.

26. The method of claim 1, wherein said DBCs are CD14⁺ monocytes.

27. The method claim 1, where the aggregation of DBCs in step (1) is performed by adding a first liquid comprising immunoglobulins, said liquid being able to lyse erythrocytes contained in the sample.

28. The method claim 1, comprising the steps of
(1a) mixing the said sample or an aliquot of the said sample with a first liquid comprising antibodies binding to other structures on the surface of other cells different from said specific sub-group of cells but carrying said CD4 receptors, forming particles or aggregates or clusters of particles or cells with a size significantly larger than the size of the cells in said specific sub-group of cells,
(1b) filter away said formed particles or aggregates or cluster of particles or cells by means of a first filter that is constituted by a size exclusion filter, and
(2) passing the remaining mixture through a second filter retaining the said specific sub-group of cells (in said sample but letting CD4 receptor molecules in solution pass through the filter, optionally followed by a washing step,
(3a) followed by exposing the said second filter to a liquid comprising labeled antibodies specifically reactive to said CD4 receptors, where said label is constituted by an enzyme or colored or fluorescent particle, optionally followed by a washing step,
(3b) optionally followed by adding a substrate to said enzyme generating a colored or fluorescent substance, and
(3c) measuring the intensity of the color or the fluorescence on said second filter and correlating said intensity to the concentration of said class of CD4 receptors on the surface of the said specific sub-group of cells

29. The method of claim 1, wherein a (selective), preferably hypotonic, lysis of erythrocytes is performed to said blood sample prior to the assessment (i.e. before step (1), (1a) and (4)) is performed.

30. The method of claim 1, wherein the cell count for the group of CD4⁺ cells is assessed.

31. The method of claim 30, wherein the cell count for the group of CD4⁺ cells, and at least for one further group of cells, different from CD4⁺ cells, in particular for the group of CD8⁺, cells is assessed, in particular the CD4/CD8 ratio.

32. A method for assessing the quantity of CD4 receptors located on the surfaces of CD4⁺ cells and optionally for assessing the quantity of CD8 receptors located on the surfaces of CD8⁺ cells in a sample of whole blood or a sample derived from blood, which method comprises performing the method of claim 1 and correlating the signal obtained for the assessment of the group of CD4⁺ cells with the quantity of cell-bound CD4⁺ receptor, and optionally correlating the signal obtained for the assessment of the group of CD8⁺ cells with the quantity of cell-bound CD8⁺ receptor.

33. (canceled)

34. (canceled)

35. A vertical flow assay device comprising:
an upper cover sheet provided with at least one circular sample feed opening and a lower absorbent layer fixed to said upper cover sheet;
a first circular filter, removably inserted into said at least one circular opening;
a second filter fixed between said upper cover sheet and said lower absorbent layer, and separating said at least one feed opening and the circular filter inserted therein from the absorbent layer.

36-43. (canceled)

44. The use of a device as claimed in claim 35 for performing an assay as claimed in claim 1.


SEQUENCE LISTING (1 sequence)

SEQ ID NO: 1 - protein, 317 aa, Homo sapiens
MNHKVHMELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPG